SEQUENCE LISTING (18 sequences)

SEQ ID NO: 1 - protein, 333 aa, Triticum aestivum
MANNGMASSPSAFFPPNFLLHMQQAPPQHDPQEHHQQHHHHHHEHHLPPPHPQHNPFLPSPQCPSLQDFRGGLSPMLGKRPAMYGGGGDGGCGGDEVTGGGANEEETSDDGSQLGGEKKRRLNVEQVRTLEKNFEVANKLEPERKMQLARALGLQPRQVAIWFQNRRARWKTKQLEKDYDVLKRQFDAVKAENDALLSHNKKLQSEILGLKECREAASELINLNKETEASCSYRSENSSEINLDISGTPPSDGPMDASGPLKHQHGRRGGGMIPFYPSVARPAGVDIDHFLHASVPKMEHHHGGPDTPSFGNLLCGVDEPPPFWPWADHQQFN

SEQ ID NO: 2 - DNA, 1126 bp, Triticum aestivum
ggaagaagccaatggccaacaatggcatggcgtcctctccctccgccttcttccctccgaatttcctcctccacatgcagcaagcaccaccgcagcatgacccccaagaacaccaccagcagcaccaccaccaccaccatgagcaccacctccctcctccccatccccagcacaaccccttcctcccctccccccaatgcccctccctccaggacttccgtggcggtctgtcgccaatgctggggaagcgcccggcgatgtacgggggcggcggcgacgggggctgcggcggcgacgaggtgaccggcggaggcgcgaacgaggaggagacgtcggacgacgggtcgcagctgggcggcgagaagaagcggcggctgaacgtggagcaggtgcggacgctggagaagaacttcgaggtggccaacaagctggagccggagcgcaagatgcagctggcccgcgcgctggggctgcagccgcggcaggtggccatctggttccagaaccgccgcgcccggtggaagacaaagcagctggagaaggactacgacgtgctcaagcgccagttcgacgccgtcaaggccgagaacgacgccctcctctcccacaacaagaagctccagtccgagatactgggactgaaggaatgcagggaggcggcgtcggagctgatcaacctcaacaaggagacggaggcgtcctgcagctaccgcagcgagaacagctctgagatcaacctcgacatctcgggcacgccgccgtccgacggccccatggacgcctccggtcctctcaagcaccagcatgggaggcgggggggggggatgatccccttctacccctccgtcgcgcgccccgcgggtgtcgacatcgaccatttcctccacgcctccgtccccaagatggagcaccaccacggcggccccgacacgcccagcttcggcaacctgctgtgcggcgtcgacgagccgccgccgttctggccgtgggcggatcaccagcagttcaactgatcgcccggtcggtcgatcgggacgatatatattttgttgcagtcaatgacgtctgctttgcttgcatggtcagcaatgagcgtgcataaatatacgatgcgtttggattcaaa

SEQ ID NO: 3 - DNA, 21 bp, Artificial
  primer
cagcttcggcaacctgctgtg

SEQ ID NO: 4 - DNA, 21 bp, Artificial
  primer
ttgccaaatgtttgaacgatc

SEQ ID NO: 5 - DNA, 21 bp, Artificial
  primer
tcattgccagagtgttgtcag

SEQ ID NO: 6 - DNA, 21 bp, Artificial
  primer
ctaggctttatttagcgatac

SEQ ID NO: 7 - DNA, 21 bp, Artificial
  primer
ttcctccccctcccgaagatc

SEQ ID NO: 8 - DNA, 21 bp, Artificial
  primer
aagaacgtatttccgctattc

SEQ ID NO: 9 - DNA, 20 bp, Artificial
  primer
gcccaagggacccattctta

SEQ ID NO: 10 - DNA, 20 bp, Artificial
  primer
ttagaacttggaacccccca

SEQ ID NO: 11 - DNA, 21 bp, Artificial
  primer
tgagcagctgccgttgcttgg

SEQ ID NO: 12 - DNA, 23 bp, Artificial
  primer
aatagtagcctacatcacctctg

SEQ ID NO: 13 - DNA, 25 bp, Artificial
  primer
acagtttggacgcaagttttgtatt

SEQ ID NO: 14 - DNA, 25 bp, Artificial
  primer
cggtcctctgttcaattcttgttta

SEQ ID NO: 15 - DNA, 22 bp, Artificial
  primer
tgtggcatcaactgctaggaaa

SEQ ID NO: 16 - DNA, 23 bp, Artificial
  primer
cgtacaaagtgcctcataggaaa

SEQ ID NO: 17 - DNA, 20 bp, Artificial
  primer
acacgagtcactgggccaga

SEQ ID NO: 18 - DNA, 23 bp, Artificial
  primer
ctggtaaactagtcacccgctga

(19) United States
(12) Patent Application Publication
(10) Pub. No.: US 2011/0321186 A1
(43) Pub. Date: Dec. 29, 2011

(54) MODULATION OF PLANT CELL WALL DEPOSITION VIA HDZIPI

(75) Inventors: Sergiy Lopato, Morphett Vale (AU); Nataliya Kovalchuk, Brompton (AU); Peter Langridge, Teringie (AU); Neil Shirley, Crafers (AU)

(73) Assignee: AUSTRALIAN CENTRE FOR PLANT FUNCTIONAL GENOMICS PTY LTD, South Australia (AU)

(21) Appl. No.: 12/998,184

(22) PCT Filed: Sep. 25, 2009
(86) PCT No.: PCT/AU2009/001273

§ 371 Date: Jun. 14, 2011

(30) Foreign Application Priority Data
Sep. 26, 2008 (AU) 2008905026

(51) Int. Cl. A01H 5/00; C12N 15/82; C12Q 1/68; C12N 5/10

(52) U.S. Cl. 800/278; 435/468; 435/419; 800/298; 435/6.12

(57) ABSTRACT

The instant invention is predicated, in part, on the functional characterization of homeodomain/leucine zipper (HDZip) polypeptides which modulate various aspects of cell wall deposition in plants, including secondary cell wall deposition. The present invention provides, among other things, methods for modulating cell wall deposition in plant cells; plant cells and plants having modulated cell wall deposition; and methods for determining and/or predicting the rate and/or extent of cell wall deposition in plant cells and plants.

### PRIORITY CLAIM

This application claims priority to Australian provisional patent application 2008905026, filed 26 Sep. 2008, the content of which is hereby incorporated by reference.

### TECHNICAL FIELD

The present invention is predicated, in part, on the functional characterisation of homeodomain/leucine zipper (HDZip) polypeptides which modulate various aspects of cell wall deposition in plant cells, including secondary cell wall deposition. The present invention provides, among other things, methods for modulating cell wall deposition in plant cells; plant cells and plants having modulated cell wall deposition; and methods for determining and/or predicting the rate and/or extent of cell wall deposition in plant cells and plants.

### BACKGROUND

Plant growth depends upon the highly coordinated processes of cell division and expansion in different tissues in response to developmental, spatial and environmental stimuli. Growth patterns in response to environmental cues allow the balancing of conflicting stimuli and processes to optimise growth responses to suit the circumstances. For example, both cell division and cell expansion are stimulated by darkness and far red light to trigger seed germination and rapid stem elongation to help the plant escape the soil or shade Conversely, white light will inhibit both these processes in the stem but will enhance cell expansion in the leaves to help optimize light capture.

Several factors are known to be closely associated with the regulation of cell expansion. Two factors are potential key regulatory triggers for both the extent and timing of cell expansion; cell turgor and cell wall plasticity. Turgor must be maintained during expansion but osmotic pressure can decline as water moves into vacuoles that help drive expansion. The main agents that hinder a decline in turgor are glucose and fructose, the products of sucrose break down. Young cells formed in meristematic regions of plants, take up water and enlarge by irreversible yielding and expanding of primary walls. Termination of cell enlargement is accompanied by the synthesis of strong, thick secondary walls.

Secondary cell wall formation involves several processes including lignin deposition, covalent crosslinking between cell wall polymers, incorporation of the glycoprotein extensin and also proteins involved in regulating cell wall structure such as chitinase-like enzymes and arabinogalactan proteins.

The genetic and biochemical processes of secondary cell wall biosynthesis have been characterized in great detail. However, information about signal transduction and transcriptional regulatory proteins, which either activate or inhibit biosynthesis of secondary cell walls, remains incomplete.

In light of the above, it would be desirable to identify transcription factors or other signal transduction molecules which may be useful for modulating the rate and/or extent of cell wall deposition in plant cells. In particular, methods which allow modulating the rate and/or extent of secondary cell wall deposition would be desirable.

Reference to any prior art in this specification is not, and should not be taken as, an acknowledgment or any form of suggestion that this prior art forms part of the common general knowledge in any country.

### SUMMARY OF THE INVENTION

The present invention is predicated, in part, on the functional characterisation of a homeodomain/leucine zipper (HDZip) polypeptide. It has been determined that expression of a HDZip polypeptide, including a class I HDZip polypeptide (HDZipI), effects promotion or enhancement of various aspects of cell wall deposition, including secondary cell wall deposition, in plant cells. Furthermore, modulation of HDZipI expression and associated modulation of cell wall deposition in one or more cells of a plant has been shown to have specific phenotypic effects on the plant.

In a first aspect, the present invention provides a method for modulating the rate and/or extent of cell wall deposition in a plant cell, the method comprising modulating the expression of a homeodomain/leucine zipper (HDZip) polypeptide in the plant cell.

In some embodiments, the HDZip polypeptide is an HDZipI polypeptide. In some embodiments, the HDZipI polypeptide is a polypeptide comprising the amino acid sequence set forth in SEQ ID NO: 1 or a functional equivalent thereof.

In some embodiments, the cell wall deposition comprises secondary cell wall deposition.

In some embodiments, an increase in the expression of an HDZipI polypeptide in the plant cell effects an increase in the rate and/or extent of cell wall deposition (including secondary cell wall deposition) in a plant cell.

In a second aspect, the present invention provides a genetically modified plant cell comprising a modulated rate and/or extent of cell wall deposition relative to an unmodified form of the cell, wherein modulation of the rate and/or extent of cell wall deposition is effected by modulation of the expression of an HDZip polypeptide in the genetically modified cell, relative to an unmodified form of the cell.

In some embodiments, the HDZip polypeptide is an HDZipI polypeptide. In some embodiments, the HDZipI polypeptide is a polypeptide comprising the amino acid sequence set forth in SEQ ID NO: 1 or a functional equivalent thereof.

In some embodiments, the cell comprises a modulated rate and/or extent of secondary cell wall deposition.

In a third aspect, the present invention provides a plant or a part, organ or tissue thereof comprising one or more cells according to the second aspect of the invention.

In some embodiments, the plant or a part, organ or tissue thereof displays exhibits an altered phenotype relative to an unmodified form of the plant.

In a fourth aspect, the present invention also provides a method for altering the phenotype of a plant, the method comprising modulating the expression of a homeodomain/leucine zipper (HDZip) polypeptide in one or more cells of the plant.

In some embodiments, the HDZip polypeptide is an HDZipI polypeptide. In some embodiments, the HDZipI polypeptide is a polypeptide comprising the amino acid sequence set forth in SEQ ID NO: 1 or a functional equivalent thereof.

In some embodiments, the phenotype of the plant is altered by increasing the expression of an HDZipI polypeptide in one or more cells of the plant.

In a fifth aspect, the present invention also provides a method for determining and/or predicting the rate and/or extent of cell wall deposition in a plant, or a part, organ, tissue or cell thereof, the method comprising determining the expression of a homeodomain/leucine zipper (HDZip) polypeptide in the plant or a part, organ, tissue or cell thereof.

In some embodiments, the HDZip polypeptide is an HDZipI polypeptide. In some embodiments, the HDZipI polypeptide is a polypeptide comprising the amino acid sequence set forth in SEQ ID NO: 1 or a functional equivalent thereof.

In some embodiments, the fifth aspect of the invention provides a method determining and/or predicting the rate and/or extent of secondary cell wall deposition in a plant, or a part, organ, tissue or cell thereof.

In some embodiments, increased expression of an HDZipI polypeptide in the plant, or a part, organ, tissue or cell thereof is indicative of an increased rate and/or extent of cell wall deposition, including secondary cell wall deposition, in the plant, or a part, organ, tissue or cell thereof.

Nucleotide and amino acid sequences are referred to herein by a sequence identifier number (SEQ ID NO:). A summary of the sequence identifiers is provided in Table 1. A sequence listing is provided at the end of the specification.

TABLE 1
Summary of Sequence Identifiers

| Sequence Identifier | Sequence |
|---|---|
| SEQ ID NO: 1 | TaHDZipI-2 amino acid sequence |
| SEQ ID NO: 2 | TaHDZipI-2 cDNA nucleotide sequence |
| SEQ ID NO: 3 | TaHDZipI-2 forward primer |
| SEQ ID NO: 4 | TaHDZipI-2 reverse primer |
| SEQ ID NO: 5 | Laccase 1 forward primer |
| SEQ ID NO: 6 | Laccase 1 reverse primer |
| SEQ ID NO: 7 | Laccase 2 forward primer |
| SEQ ID NO: 8 | Laccase 2 reverse primer |
| SEQ ID NO: 9 | HvCesA4 forward primer |
| SEQ ID NO: 10 | HvCesA4 reverse primer |
| SEQ ID NO: 11 | HvCesA7 forward primer |
| SEQ ID NO: 12 | HvCesA7 reverse primer |
| SEQ ID NO: 13 | HvCesA8 forward primer |
| SEQ ID NO: 14 | HvCesA8 reverse primer |
| SEQ ID NO: 15 | HvCesA1 forward primer |
| SEQ ID NO: 16 | HvCesA1 reverse primer |
| SEQ ID NO: 17 | HvCesA3 forward primer |
| SEQ ID NO: 18 | HvCesA3 reverse primer |

### DESCRIPTION OF EXEMPLARY EMBODIMENTS

It is to be understood that the following description is for the purpose of describing particular embodiments only and is not intended to be limiting with respect to the above description.

In a first aspect, the present invention provides a method for modulating the rate and/or extent of cell wall deposition in a plant cell, the method comprising modulating the expression of a homeodomain/leucine zipper (HDZip) polypeptide in the plant cell.

Plant cells are typically enclosed by a cell wall containing cellulose. The cell wall has a number of functions: it lends the cell stability, it determines its shape, influences its development, protects the cell against pathogens and counterbalances osmotic pressure. The cell wall of elongating cells is elastic, a property which is generally lost in fully differentiated cells.

Cell walls can be classified as primary or secondary walls. The primary cell wall is laid out during the first division of the cell. It develops normally between the two daughter cells during early telophase.

The early stage of the new cell wall is the cell plate, a lamella-like structure in the former equatorial plane of the mitotic apparatus. Electron microscopic studies show that it develops by fusion of numerous vesicles. The plate grows centrifugally until it reaches the longitudinal lateral walls of the mother cell. Electron dense material is deposited at both its sides. The thus developing structure is called the phragmoplast. It is the immediate precursor of the primary wall.

Primary cell walls are generally deposited during cell wall expansion or elongation and are composed mostly of polysaccharides (approx 90%) such as cellulose, pectin, heteroxylans, xyloglucans, 1-3,1-4-β glucans and/or mannans. Primary cell walls may also contain approximately 5-10% proteins including both structural and enzymatic proteins. Primary cell walls may also contain phenolic compounds.

As set out above, the disclosed method contemplates modulating the rate and/or extent of cell wall deposition in a plant cell.

In some embodiments, reference herein to “cell wall deposition” should be understood to refer to secondary cell wall deposition.

The term “secondary cell wall”, as used herein, generally refers to cell wall material which is deposited after cessation of cell wall expansion. The secondary wall develops by successive encrustation and deposition of cellulose fibrils and other components on the inside of the primary cell wall. Secondary cell wall deposition generally occurs when the cell has stopped growth and wall elasticity is no longer required. While the primary wall structure is generally similar across plant cell types and species, there are cell type and species-specific differences typical for the secondary cell wall.

The most striking feature of secondary walls is their loss of plasticity. Progressive depositions of new lamellas thicken the wall while the cell lumen's diameter decreases. Secondary cell walls are generally less hydrated than primary walls and contain less pectins and hemicellulose. Instead other components are deposited, which are sometimes characteristic for certain cell groups or tissues.

Secondary cell walls can also be lignified. Lignin is the basic unit of xylem and strengthening elements (wood) and consists of polymerized phenylpropane units. The three most important starting compounds are coumaryl alcohol (with an OH-group in position 4 of the phenyl ring), coniferyl alcohol (OH-group in position 4, —OCH₃ in position 3) and sinapyl alcohol (OH-group in position 4, —OCH₃ group in positions 3 and 5). The lignins of plant groups differ in the percentages of these starting compounds and in the way they are linked. All bonds leading to the formation of a three-dimensional molecular network are covalent. As a consequence lignins form a network that provides stability. However, the bonds are irreversible, and stretching of the wall and growth of the cell are generally impossible after substantial wall lignification. The lignin of pteridophytes consists mainly of coniferyl alcohol polymers, while in dicots coniferyl and sinapyl alcohol polymers occur in roughly equal amounts. In the lignins of all plant groups are only trace amounts of coumaryl alcohol are found.

Mannans may also be incorporated into secondary walls, and are a structural element of many seeds. The secondary walls of pollen also contain sporopollenin, a polymerization product of carotene.

Many secondary walls also contain a wide range of strongly hydrophobic compounds, like suberine, the basic component of cork. Such compounds may comprise integral components of the wall itself. Alternatively, such compounds may be deposited on the wall as solid excretion products (cuticle, wax deposits, etc.).

Beside the structural elements of the wall, non-structural components may also be part of the secondary cell wall. These components may include a number of low molecular weight compounds (dyes, alcohols, terpenes, tannins, etc.), oligosaccharides (and polysaccharides) of different configurations as well as proteins (usually glycoproteins). Some of them participate in recognition processes, such as incompatibility factors at the stigma surface and several carbohydrate-binding lectins.

As set out above, the present invention is predicated, in part, on modulating the rate and/or extent of cell wall deposition in a plant cell.

As referred to herein, “modulation” of the rate and/or extent of cell wall deposition in a plant cell should be understood to include an increase or decrease in the rate and/or extent of cell wall deposition in a plant cell.

By “increasing” is intended, for example, a 1%, 5%, 10%, 20%, 30%, 40%, 50%, 60%, 70%, 80%, 90%, 100%, 2-fold, 5-fold, 10-fold, 20 fold, 50-fold, 100-fold increase in the rate and/or extent of cell wall deposition in the plant cell. By “decreasing” is intended, for example, a 1%, 5%, 10%, 20%, 30%, 40%, 50%, 60%, 70%, 80%, 90%, 91%, 92%, 93%, 94%, 95%, 96%, 97%, 98%, 99%, 100% reduction in the rate and/or extent of cell wall deposition in the plant cell.

“Modulating” should also be understood to include introducing cell wall deposition into a plant cell which does not have a primary and/or secondary cell wall, such as protoplasts, some algae and the like. In addition, “modulating” may also include the substantially complete inhibition of primary and/or secondary cell wall deposition in a plant cell.

As set out above, the disclosed method contemplates modulating the expression of a “homeodomain/leucine zipper (HDZip) polypeptide” in the plant cell.

“HDZip polypeptides” include polypeptides that comprise both homeodomain and leucine zipper structural motifs.

The homeodomain motif is a protein structural domain that binds DNA and is thus commonly found in transcription factors. The motif consists of a 60-amino acid helix-turn-helix structure in which three alpha helices are connected by short loop regions. The N-terminal two helices are antiparallel and the longer C-terminal helix is roughly perpendicular to the axes established by the first two.

Genetic and structural analyses of the homeodomain suggest a general model for homeodomain binding to DNA, in which the most highly conserved of three a-helices (helix 3) fits directly into the major groove of DNA.

Homeodomains can bind both specifically and nonspecifically to B-DNA with the C-terminal recognition helix aligning in the DNA's major groove and the unstructured peptide “tail” at the N-terminus aligning in the minor groove. The recognition helix and the inter-helix loops are rich in arginine and lysine residues, which form hydrogen bonds to the DNA backbone; conserved hydrophobic residues in the center of the recognition helix aid in stabilizing the helix packing. Homeodomain proteins show a preference for the DNA sequence 5′-ATTA-3′; while sequence-independent binding occurs with significantly lower affinity.

HDZip polypeptides also comprise a leucine zipper structural motif in addition to a homeodomain structural motif.

The main feature of the leucine zipper motif is the predominance of the common amino acid leucine at the d position of a heptad repeat. Leucine zippers were first identified by sequence alignment of certain transcription factors which identified a common pattern of leucines every seven amino acids. These leucines were later shown to form the hydrophobic core of a coiled coil. Each half of a leucine zipper consists of a short alpha-helix with a leucine residue at every seventh position. The standard 3.6 residues per turn alpha-helix structure changes slightly to become a 3.5 residues per turn alpha-helix. In this structure, one leucine comes in direct contact with another leucine on the other strand every second turn.

HDZip polypeptides may be classified into the HDZipI, HDZipII, HDZipIII, and HDZipIV subfamilies. For details of the classification of HDZip polypeptides into the various subfamilies, see Meijer et al. (Plant J. 11: 263-276, 1997) and Aso et al. (Mol. Biol. Evol. 16: 544-551, 1999).

The functions of the HDZip genes are diverse among the different subfamilies, and even within the same subfamily. For example, HDZipI and II genes have been demonstrated to be involved in the signal transduction networks of light, dehydration-induced ABA and auxin. These signal transduction networks are related to the general growth regulation of plants. Members of the HDZipIII subfamily have been shown to play roles in cell differentiation in the stele, although the functions of some genes remain unknown. HDZipIV genes have been shown to be related to the differentiation of the outermost cell layer.

In some embodiments described herein, the HDZip polypeptide is a class I HDZip polypeptide (HDZipI polypeptide).

In some embodiments, the HDZipI polypeptide comprises a polypeptide comprising the amino acid sequence set forth in SEQ ID NO: 1 or a functional equivalent thereof.

A “functional equivalent” of a polypeptide which comprises the amino acid sequence set forth in SEQ ID NO: 1 should be understood as an HDZipI polypeptide which has the function of upregulating secondary cell wall deposition, as described herein.

In some embodiments, the functional equivalent comprises a polypeptide which comprises an amino acid sequence which is at least 50% identical to SEQ ID NO: 1.

As such, the functional equivalent may be, for example, a polypeptide which has one or more amino acid insertions, deletions or substitutions relative to the polypeptide comprising the amino acid sequence set forth in SEQ ID NO: 1; a mutant form or allelic variant of the polypeptide comprising the amino acid sequence set forth in SEQ ID NO: 1; an ortholog of the polypeptide comprising the amino acid sequence set forth in SEQ ID NO: 1; an analog of the polypeptide comprising the amino acid sequence set forth in SEQ ID NO: 1; and the like.

In some embodiments, reference herein to “at least 50%” sequence identity with regard to SEQ ID NO: 1, should be understood to encompass higher levels of sequence identity, including at least 60% amino acid sequence identity, at least 70% amino acid sequence identity, at least 80% amino acid sequence identity, at least 85% amino acid sequence identity, at least 90% amino acid sequence identity or at least 95%, 96%, 97%, 98%, 99% or 100% amino acid sequence identity to SEQ ID NO: 1.

When comparing amino acid sequences, the compared sequences should be compared over a comparison window of at least 50 amino acid residues, at least 100 amino acid residues, at least 200 amino acid residues, at least 250 amino acid residues or over the full length of SEQ ID NO: 1. The comparison window may comprise additions or deletions (ie. gaps) of about 20% or less as compared to the reference sequence (which does not comprise additions or deletions) for optimal alignment of the two sequences. Optimal alignment of sequences for aligning a comparison window may be conducted by computerized implementations of algorithms such the BLAST family of programs as, for example, disclosed by Altschul et al. (Nucl. Acids Res. 25: 3389-3402, 1997). A detailed discussion of sequence analysis can be found in Unit 19.3 of Ausubel et al. (Current Protocols in Molecular Biology, John Wiley & Sons Inc, 1994-1998, Chapter 15, 1998).

Examples of “functional equivalents” of a polypeptide comprising the amino acid sequence set forth in SEQ ID NO: 1 include polypeptides encoded by any of Hox21 (eg. NCBI accession numbers AY554028 and EF555544), Hox23 (eg. NCBI accession number EU085431), or AtHB13 (eg. NCBI accession number NM_{—}102460, NM_{—}105646 and AF208044).

The present invention contemplates any means by which the expression of an HDZip polypeptide in a cell may be modulated. This includes, for example, methods such as the application of agents which modulate HDZip polypeptide activity in a cell, including the application of a HDZip polypeptide agonist or antagonist; the application of agents which mimic HDZip polypeptide activity in a cell; modulating the expression of a HDZip polypeptide encoding nucleic acid in the cell; or effecting the expression of an altered or mutated HDZip polypeptide encoding nucleic acid in a cell such that a HDZip polypeptide with increased or decreased specific activity, half-life and/or stability is expressed by the cell.

In some embodiments, the expression of the HDZip polypeptide is modulated by modulating the expression of an HDZip polypeptide encoding nucleic acid in the cell.

The term “modulating” with regard to the expression of an HDZip polypeptide encoding nucleic acid may include increasing or decreasing the transcription and/or translation of an HDZip polypeptide encoding nucleic acid in the cell. By “increasing” is intended, for example a 1%, 5%, 10%, 20%, 30%, 40%, 50%, 60%, 70%, 80%, 90%, 100%, 2-fold, 5-fold, 10-fold, 20-fold, 50-fold, 100-fold or greater increase in the transcription and/or translation of a HDZip polypeptide encoding nucleic acid. By “decreasing” is intended, for example, a 1%, 5%, 10%, 20%, 30%, 40%, 50%, 60%, 70%, 80%, 90%, 91%, 92%, 93%, 94%, 95%, 96%, 97%, 98%, 99%, 100% reduction in the transcription and/or translation of a HDZip polypeptide encoding nucleic acid. Modulating also comprises introducing expression of an HDZip polypeptide encoding nucleic acid not normally found in a particular cell; or the substantially complete inhibition (eg. knockout) of expression of an HDZip polypeptide encoding nucleic acid in a cell that normally has such activity.

As referred to herein, an “HDZip polypeptide encoding nucleic acid” refers to any nucleic acid which encodes an HDZip polypeptide, as hereinbefore described. In some embodiments, the HDZip polypeptide encoding nucleic acid encodes a class I HDZip polypeptide.

In some embodiments, an HDZipI polypeptide-encoding nucleic acid comprises the nucleotide sequence set forth in SEQ ID NO: 2 or a nucleotide sequence which is at least 50% identical thereto.

The HDZipI polypeptide-encoding nucleic acid having the defined level of sequence identity with SEQ ID NO: 2 may be a nucleic acid which has one or more nucleotide insertions, deletions or substitutions relative to the nucleic acid comprising the nucleotide sequence set forth in SEQ ID NO: 2; a mutant form or allelic variant of the nucleotide sequence set forth in SEQ ID NO: 2; an ortholog of the nucleotide sequence set forth in SEQ ID NO: 2; and the like.

Reference herein to “at least 50%” sequence identity with regard to SEQ ID NO: 2, in some embodiments at least to encompass higher levels of sequence identity, including at least 60% amino acid sequence identity, at least 70% amino acid sequence identity, at least 80% amino acid sequence identity, at least 85% amino acid sequence identity, at least 90% amino acid sequence identity or at least 95%, 96%, 97%, 98%, 99% or 100% amino acid sequence identity to SEQ ID NO: 1.

When comparing nucleotide sequences, the compared sequences should be compared over a comparison window of at least 100 nucleotide residues, at least 200 nucleotide residues, at least 300 nucleotide residues, at least 400 nucleotide residues, at least 500 nucleotide residues, at least 600 nucleotide residues, at least 800 nucleotide residues, at least 1000 nucleotide residues, or over the full length of SEQ ID NO: 2. The comparison window may comprise additions or deletions (ie. gaps) of about 20% or less as compared to the reference sequence (which does not comprise additions or deletions) for optimal alignment of the two sequences. Optimal alignment of sequences for aligning a comparison window may be conducted by computerized implementations of algorithms such the BLAST family of programs as, for example, disclosed by Altschul et al. (Nucl. Acids Res. 25: 3389-3402, 1997). A detailed discussion of sequence analysis can be found in Unit 19.3 of Ausubel et al. (Current Protocols in Molecular Biology, John Wiley & Sons Inc, 1994-1998, Chapter 15, 1998).

The present invention contemplates any means by which the expression of an HDZip polypeptide encoding nucleic acid may be modulated. For example, exemplary methods for modulating the expression of a HDZip polypeptide encoding nucleic acid include, for example: genetic modification of the cell to upregulate or downregulate expression of an endogenous HDZip polypeptide encoding nucleic acid; genetic modification by transformation with a HDZip polypeptide encoding nucleic acid; genetic modification to increase the copy number of a HDZip polypeptide encoding nucleic acid sequence in the cell; administration of a nucleic acid molecule to the cell which modulates expression of an endogenous HDZip polypeptide encoding nucleic acid in the cell; and the like.

In some embodiments, the expression of an HDZip polypeptide encoding nucleic acid is modulated by genetic modification of the cell. The term “genetically modified”, as used herein, should be understood to include any genetic modification that effects an alteration in the expression of an HDZip polypeptide encoding nucleic acid in the genetically modified cell relative to a non-genetically modified form of the cell. Exemplary types of genetic modification include: random mutagenesis such as transposon, chemical, UV and phage mutagenesis together with selection of mutants which overexpress or underexpress an endogenous HDZip polypeptide encoding nucleic acid; transient or stable introduction of one or more nucleic acid molecules into a cell which direct the expression and/or overexpression of HDZip polypeptide encoding nucleic acid in the cell; inhibition of an endogenous HDZip polypeptide encoding nucleic acid by site-directed mutagenesis of an endogenous HDZip polypeptide encoding nucleic acid; introduction of one or more nucleic acid molecules which inhibit the expression of an endogenous HDZip polypeptide encoding nucleic acid in the cell, eg. a cosuppression construct or an RNAi construct; and the like.

In some embodiments, the present invention contemplates increasing the level of HDZip polypeptide in a cell, by introducing the expression of an HDZip polypeptide encoding nucleic acid into the cell, upregulating the expression of an HDZip polypeptide encoding nucleic acid in the cell and/or increasing the copy number of an HDZip polypeptide encoding nucleic acid in the cell. In some embodiments, the introduced HDZip polypeptide encoding nucleic acid may be placed under the control of a transcriptional control sequence such as a native promoter or a heterologous promoter.

In some embodiments, an increase in the expression of an HDZipI polypeptide in the plant cell effects an increase in the rate and/or extent of cell wall deposition in a plant cell.

Methods for plant transformation and expression of an introduced nucleotide sequence are well known in the art, and the present invention contemplates the use of any suitable method.

Suitable methods for the transformation of plant cells include, for example: Agrobacterium-mediated transformation, microprojectile bombardment based transformation methods and direct DNA uptake based methods. Roa-Rodriguez et al. (Agrobacterium-mediated transformation of plants, 3rd Ed. CAMBIA Intellectual Property Resource, Canberra, Australia, 2003) review a wide array of suitable Agrobacterium-mediated plant transformation methods for a wide range of plant species. Bacterial mediated transformation using bacteria other than Agrobacterium sp. may also be used, for example as described in Broothaerts et al. (Nature 433: 629-633, 2005). Microprojectile bombardment may also be used to transform plant tissue and methods for the transformation of plants, including cereal plants, and such methods are reviewed by Casas et al. (Plant Breeding Rev. 13: 235-264, 1995). Direct DNA uptake transformation protocols such as protoplast transformation and electroporation are described in detail in Galbraith et al. (eds.), Methods in Cell Biology Vol. 50, Academic Press, San Diego, 1995). In addition to the methods mentioned above, a range of other transformation protocols may also be used. These include infiltration, electroporation of cells and tissues, electroporation of embryos, microinjection, pollen-tube pathway, silicon carbide- and liposome mediated transformation. Methods such as these are reviewed by Rakoczy-Trojanowska (Cell. Mol. Biol. Lett. 7: 849-858, 2002). A range of other plant transformation methods may also be evident to those of skill in the art. By way of further example, reference is also made to Zhao et al. (Mol Breeding DOI 10.1007/s11032-006-9005-6, 2006), Katsuhara et al. (Plant Cell Physiol 44(12): 1378-1383, 2003), Ohta et al. (FEBS Letters 532: 279-282, 2002) and Wu et al. (Plant Science 169: 65-73, 2005).

In some embodiments the present invention also provides methods for down-regulating expression of an HDZip polypeptide encoding nucleic acid in a cell.

In some embodiments, a decrease in the expression of an HDZipI polypeptide in the plant cell effects a decrease in the rate and/or extent of secondary cell wall deposition in a plant cell.

The present invention contemplates methods such as knockout or knockdown of an endogenous HDZip polypeptide encoding nucleic acid in a cell using methods including, for example:
insertional mutagenesis of a HDZip polypeptide encoding nucleic acid in a cell including knockout or knockdown of a HDZip polypeptide encoding nucleic acid in a cell by homologous recombination with a knockout construct (for an example of targeted gene disruption in plants see Terada et al., Nat. Biotechnol. 20: 1030-1034, 2002) or by T-DNA or transposon mutagenesis.
post-transcriptional gene silencing (PTGS) or RNAi of an HDZip polypeptide encoding nucleic acid in a cell (for review of PTGS and RNAi see Sharp, Genes Dev. 15(5): 485-490, 2001; and Hannon, Nature 418: 244-51, 2002);
transformation of a cell with an antisense construct directed against a HDZip polypeptide encoding nucleic acid (for examples of antisense suppression in plants see van der Krol et al., Nature 333: 866-869; van der Krol et al., BioTechniques 6: 958-967; and van der Krol et al., Gen. Genet. 220: 204-212);
transformation of a cell with a co-suppression construct directed against an HDZip polypeptide encoding nucleic acid (for an example of co-suppression in plants see van der Krol et al., Plant Cell 2(4): 291-299);
transformation of a cell with a construct encoding a double stranded RNA directed against an HDZip polypeptide encoding nucleic acid (for an example of dsRNA mediated gene silencing see Waterhouse et al., Proc. Natl. Acad. Sci. USA 95: 13959-13964, 1998); and
transformation of a cell with a construct encoding an siRNA or hairpin RNA directed against an HDZip polypeptide encoding nucleic acid (for an example of siRNA or hairpin RNA mediated gene silencing in plants see Lu et al., Nucl. Acids Res. 32(21): e171; doi:10.1093/nar/gnh170, 2004).

The present invention also facilitates the downregulation of an HDZip polypeptide encoding nucleic acid in a cell via the use of synthetic oligonucleotides, for example, siRNAs or microRNAs directed against a HDZip polypeptide encoding nucleic acid (for examples of synthetic siRNA mediated silencing see Caplen et al., Proc. Natl. Acad. Sci. USA 98: 9742-9747, 2001; Elbashir et al., Genes Dev. 15: 188-200, 2001; Elbashir et al., Nature 411: 494-498, 2001; Elbashir et al., EMBO J. 20: 6877-6888, 2001; and Elbashir et al., Methods 26: 199-213, 2002).

In addition to the examples above, the introduced nucleic acid may also comprise a nucleotide sequence which is not directly related to an HDZip polypeptide encoding nucleic acid but, nonetheless, may directly or indirectly modulate the expression of an HDZip polypeptide encoding nucleic acid in a cell. Examples include nucleic acid molecules that encode transcription factors or other proteins which promote or suppress the expression of an endogenous HDZip polypeptide encoding nucleic acid in a cell; and other non-translated RNAs which directly or indirectly promote or suppress endogenous HDZip polypeptide encoding nucleic acid expression and the like.

In order to effect expression of an introduced nucleic acid in a genetically modified cell, where appropriate, the introduced nucleic acid may be operably connected to one or more transcriptional control sequences and/or promoters.

For the purposes of the present specification, a transcriptional control sequence is regarded as “operably connected” to a given gene or other nucleotide sequence when the transcriptional control sequence is able to promote, inhibit or otherwise modulate the transcription of the gene or other nucleotide sequence.

A promoter may regulate the expression of an operably connected nucleotide sequence constitutively, or differentially, with respect to the cell, tissue, organ or developmental stage at which expression occurs, in response to external stimuli such as physiological stresses, pathogens, or metal ions, amongst others, or in response to one or more transcriptional activators. As such, the promoter used in accordance with the methods of the present invention may include, for example, a constitutive promoter, an inducible promoter, a tissue-specific promoter or an activatable promoter.

Plant constitutive promoters typically direct expression in nearly all tissues of a plant and are largely independent of environmental and developmental factors. Examples of constitutive promoters that may be used in accordance with the present invention include plant viral derived promoters such as the Cauliflower Mosaic Virus 35S and 19S (CaMV 35S and CaMV 19S) promoters; bacterial plant pathogen derived promoters such as opine promoters derived from Agrobacterium spp., eg. the Agrobacterium-derived nopaline synthase (nos) promoter; and plant-derived promoters such as the rubisco small subunit gene (rbcS) promoter, the plant ubiquitin promoter (Pubi) and the rice actin promoter (Pact).

“Inducible” promoters include, but are not limited to, chemically inducible promoters and physically inducible promoters. Chemically inducible promoters include promoters which have activity that is regulated by chemical compounds such as alcohols, antibiotics, steroids, metal ions or other compounds. Examples of chemically inducible promoters include: alcohol regulated promoters (eg. see European Patent 637 339); tetracycline regulated promoters (eg. see U.S. Pat. No. 5,851,796 and U.S. Pat. No. 5,464,758); steroid responsive promoters such as glucocorticoid receptor promoters (eg. see U.S. Pat. No. 5,512,483), estrogen receptor promoters (eg. see European Patent Application 1 232 273), ecdysone receptor promoters (eg. see U.S. Pat. No. 6,379,945) and the like; metal-responsive promoters such as metallothionein promoters (eg. see U.S. Pat. No. 4,940,661, U.S. Pat. No. 4,579,821 and U.S. Pat. No. 4,601,978); and pathogenesis related promoters such as chitinase or lysozyme promoters (eg. see U.S. Pat. No. 5,654,414) or PR protein promoters (eg. see U.S. Pat. No. 5,689,044, U.S. Pat. No. 5,789,214, Australian Patent 708850, U.S. Pat. No. 6,429,362).

The inducible promoter may also be a physically regulated promoter which is regulated by non-chemical environmental factors such as temperature (both heat and cold), light and the like. Examples of physically regulated promoters include heat shock promoters (eg. see U.S. Pat. No. 5,447,858, Australian Patent 732872, Canadian Patent Application 1324097); cold inducible promoters (eg. see U.S. Pat. No. 6,479,260, U.S. Pat. No. 6,184,443 and U.S. Pat. No. 5,847,102); light inducible promoters (eg. see U.S. Pat. No. 5,750,385 and Canadian Patent 132 1563); light repressible promoters (eg. see New Zealand Patent 508103 and U.S. Pat. No. 5,639,952).

“Tissue specific promoters” include promoters which are preferentially or specifically expressed in one or more specific cells, tissues or organs in an organism and/or one or more developmental stages of the organism. It should be understood that a tissue specific promoter may also be inducible.

Examples of plant tissue specific promoters include: root specific promoters such as those described in US Patent Application 2001047525; fruit specific promoters including ovary specific and receptacle tissue specific promoters such as those described in European Patent 316 441, U.S. Pat. No. 5,753,475 and European Patent Application 973 922; and seed specific promoters such as those described in Australian Patent 612326 and European Patent application 0 781 849 and Australian Patent 746032.

The promoter may also be a promoter that is activatable by one or more transcriptional activators, referred to herein as an “activatable promoter”. For example, the activatable promoter may comprise a minimal promoter operably connected to an Upstream Activating Sequence (UAS), which comprises, inter alia, a DNA binding site for one or more transcriptional activators.

As referred to herein the term “minimal promoter” should be understood to include any promoter that incorporates at least an RNA polymerase binding site and, optionally a TATA box and transcription initiation site and/or one or more CAAT boxes. In some embodiments wherein the cell is a plant cell, the minimal promoter may be derived from the Cauliflower Mosaic Virus 35S (CaMV 35S) promoter. The CaMV 35S derived minimal promoter may comprise, for example, a sequence that substantially corresponds to positions −90 to +1 (the transcription initiation site) of the CaMV 35S promoter (also referred to as a −90 CaMV 35S minimal promoter), −60 to +1 of the CaMV 35S promoter (also referred to as a −60 CaMV 35S minimal promoter) or −45 to +1 of the CaMV 35S promoter (also referred to as a −45 CaMV 35S minimal promoter).

As set out above, the activatable promoter may comprise a minimal promoter fused to an Upstream Activating Sequence (UAS). The UAS may be any sequence that can bind a transcriptional activator to activate the minimal promoter. Exemplary transcriptional activators include, for example: yeast derived transcription activators such as Gal4, Pdr1, Gcn4 and Ace1; the viral derived transcription activator, VP16; Hap1 (Hach et al., J Biol Chem 278: 248-254, 2000); Gaf1 (Hoe et al., Gene 215(2): 319-328, 1998); E2F (Albani et al., J Biol Chem 275: 19258-19267, 2000); HAND2 (Dai and Cserjesi, J Biol Chem 277: 12604-12612, 2002); NRF-1 and EWG (Herzig et al., J Cell Sci 113: 4263-4273, 2000); P/CAF (Itoh et al., Nucl Acids Res 28: 4291-4298, 2000); MafA (Kataoka et al., J Biol Chem 277: 49903-49910, 2002); human activating transcription factor 4 (Liang and Hai, J Biol Chem 272: 24088-24095, 1997); Bcl10 (Liu et al., Biochem Biophys Res Comm 320(1): 1-6, 2004); CREB-H (Omori et al., Nucl Acids Res 29: 2154-2162, 2001); ARR1 and ARR2 (Sakai et al., Plant J 24(6): 703-711, 2000); Fos (Szuts and Bienz, Proc Natl Acad Sci USA 97: 5351-5356, 2000); HSF4 (Tanabe et al., J Biol Chem 274: 27845-27856, 1999); MAML1 (Wu et al., Nat Genet 26: 484-489, 2000).

In some embodiments, the UAS comprises a nucleotide sequence that is able to bind to at least the DNA-binding domain of the GAL4 transcriptional activator. UAS sequences, which can bind transcriptional activators that comprise at least the GAL4 DNA binding domain, are referred to herein as UAS_{G}. In another embodiment, the UAS_{G} comprises the sequence 5′-CGGAGTACTGTCCTCCGAG-3′ or a functional homolog thereof.

As referred to herein, a “functional homolog” of the UAS_{G} sequence should be understood to refer to any nucleotide sequence which can bind at least the GAL4 DNA binding domain and which may comprise a nucleotide sequence having at least 50% identity, at least 65% identity, at least 80% identity or at least 90% identity with the UAS_{G} nucleotide sequence.

The UAS sequence in the activatable promoter may comprise a plurality of tandem repeats of a DNA binding domain target sequence. For example, in its native state, UAS_{G} comprises four tandem repeats of the DNA binding domain target sequence. As such, the term “plurality” as used herein with regard to the number of tandem repeats of a DNA binding domain target sequence should be understood to include, for example, at least 2 tandem repeats, at least 3 tandem repeats or at least 4 tandem repeats.

The transcriptional control sequence to which the HDZip encoding nucleic acid is connected may be introduced into the cell with the HDZip encoding nucleic acid itself, or alternatively, the HDZip encoding nucleic acid may be inserted into the genome of the plant cell such that it becomes operably connected to an endogenous transcriptional control sequence. In the latter embodiments, the insertion of the HDZip encoding nucleic acid in the genome such that it is under the control of an endogenous transcriptional control sequence may be the result of either non-site directed or random DNA insertion (eg. T-DNA or transposon mediated insertion) or the result of site-directed insertion (for example as described in (Terada et al., Nat. Biotechnol. 20: 1030-1034, 2002).

As set out above, the disclosed method provides a method for modulating the rate and/or extent of cell wall deposition in a plant cell. As referred to herein, the “rate and/or extent of cell wall deposition” should be understood to include, but not be limited to the actual rate and/or extent of cell wall deposition by a plant cell. The “rate and/or extent of cell wall deposition” should also be understood to include any process in the plant cell which is involved in or associated with cell wall deposition. For example, and as discussed later, modulation of the rate and/or extent of cell wall deposition may include any one or more of: modulation of actual cell wall deposition, modulation of the expression of cell wall associated proteins, modulation of the amount of one or more primary or secondary cell wall components in the plant cell wall, and the like.

Furthermore, modulation of the rate or extent of cell wall deposition should be understood to include, for example, modulation of the rate and/or extent of cell wall production or degradation in the plant cell.

In some embodiments, modulating the rate and/or extent of cell wall deposition in the plant cell comprises modulating the expression of one or more cell wall associated proteins in the plant cell.

As referred to herein, the “modulating the expression of one or more cell wall associated proteins” should be understood to include any process that effects the level and/or activity of one or more cell wall associated proteins in a plant cell. In some embodiments, however, this term should be understood to encompass modulation of the transcription and/or translation of a nucleic acid which encodes a cell wall associated protein.

Thus, also provided is a method for modulating the expression of one or more cell wall associated proteins in a plant cell, the method comprising modulating the expression of a homeodomain/leucine zipper (HDZip) polypeptide in the plant cell.

Exemplary “secondary cell wall associated proteins” include, for example, cellulose synthases, xylan synthases and other polysaccharide synthases, peroxidases, laccases, transcription factors involved in cell wall biosynthesis signaling, polysaccharide transfereases, Cobra proteins, Fla proteins and the like.

Specific examples of “secondary cell wall associated proteins” include those proteins described hereafter in Table 4.

In some embodiments, the one or more cell wall associated proteins comprise a cellulose synthase or cellulose synthase like enzyme.

Cellulose synthases may be encoded by cellulose synthase (CesA) genes, while cellulose synthase like enzymes may be encoded by the Csl gene family. Both the CesA genes and the cellulose synthase-like (Csl) gene family form a large gene superfamily.

The Csl gene superfamily, inclusive of the CesA gene family, is described in detail in the literature. In this regard, reference is made to Burton et al. (Plant Physiol. 134(1): 224-236, 2004), Richmond and Somerville (Plant Physiol. 124: 495-498, 2000) and Burton et al. (Science 311 (5769): 1940-2, 2006).

In some embodiments an increase in the expression of an HDZipI polypeptide in the plant cell effects an increase in the expression of one or more secondary cell wall associated proteins.

Examples of secondary cell wall associated proteins that may be upregulated in response to HDZipI include: cellulose synthases such as CesA4, CesA7 and CesA8; laccases such as LAC1; MYB transcription factors such as MYB1, MYB33L, MYB4, MYB54, MYBA; NAC transcription factors such as NST1 and VND6; other transcription factors such as KN7; COBRA proteins such as COBRAS; FLA proteins such as FLA10G2; and XETs such as XET2.

In some embodiments, a decrease in the expression of an HDZipI polypeptide in the plant cell effects a decrease in the expression of one or more cell wall associated proteins.

In some embodiments, modulating the rate and/or extent of cell wall deposition in the plant cell may also comprise modulating the rate and/or extent of lignin deposition in the cell wall of the plant cell.

As referred to herein, “modulating the rate and/or extent of lignin deposition in the cell wall of a plant cell” refers to increasing or decreasing the rate and/or extent of lignin deposition in the cell wall of a plant cell.

In some embodiments an increase in the expression of an HDZipI polypeptide in the plant cell may effect an increase in the rate and/or extent of lignin deposition in the cell wall of the plant cell. In some embodiments, a decrease in the expression of an HDZipI polypeptide in the plant cell may effect a decrease in the rate and/or extent of lignin deposition in the cell wall of the plant cell.

In the context of a plant cell in a whole plant or part thereof, expression of an HDZipI polypeptide in the plant may increase the rate and/or extent of lignin deposition in one more specific cell types while having no effect on other cell types or even reducing the rate and/or extent of lignin deposition in one or more cell types. Thus, in some embodiments, modulating the rate and/or extent of lignin deposition in the cell wall of a plant cell may also encompass modulating the pattern of lignin deposition in a plant comprising one or more plant cells.

Expression of an HDZip polypeptide may also affect cell size and morphology. Without limiting the present invention to any particular mode of action, it is postulated that increased expression of an HDZipI polypeptide in a plant cell may promote secondary cell wall deposition such that the period of cell elongation is shortened. Furthermore, it is also considered that the opposite would occur in that a decrease in, or inhibition of, the expression of an HDZipI polypeptide in a cell would inhibit secondary cell wall deposition and thus promote cell elongation.

As set out above, the disclosed method is practiced on a plant cell. As referred to herein a “plant cell” includes any cell from an organism of the kingdom Plantae. As such, the cell may be a bryophyte cell or a vascular plant cell. Generally, the cells used in accordance with the present invention include walled members of this kingdom. However, naturally non-walled members of the kingdom may be used and the present invention may be used to promote cell wall deposition in such cells.

In some embodiments, the cell is a monocotyledonous or dicotyledonous angiosperm plant cell or a gymnosperm plant cell. In some embodiments, the cell is a monocotyledonous plant cell, and in some embodiments a cereal crop plant cell.

As used herein, the term “cereal crop plant” includes members of the Poales (grass family) that produce edible grain for human or animal food. Examples of Poales cereal crop plants which in no way limit the present invention include wheat, rice, maize, millets, sorghum, rye, triticale, oats, barley, teff, wild rice, spelt and the like. However, the term cereal crop plant should also be understood to include a number of non-Poales species that also produce edible grain and are known as the pseudocereals, such as amaranth, buckwheat and quinoa.

In some embodiments, the cell is a barley cell. As referred to herein, “barley” includes several members of the genus Hordeum. The term “barley” encompasses cultivated barley including two-row barley (Hordeum distichum), four-row barley (Hordeum tetrastichum) and six-row barley (Hordeum vulgare). In some embodiments, barley may also refer to wild barley, (Hordeum spontaneum). In some embodiments, the term “barley” refers to barley of the species Hordeum vulgare.

Although cereal crop plants are suitable monocotyledonous plants, the other monocotyledonous plants may also be used, such as other non-cereal plants of the Poales, specifically including pasture grasses such as Lolium spp.

In a second aspect, the present invention provides a genetically modified plant cell comprising a modulated rate and/or extent of cell wall deposition relative to an unmodified form of the cell, wherein modulation of the rate and/or extent of cell wall deposition is effected by modulation of the expression of an HDZip polypeptide in the genetically modified cell, relative to an unmodified form of the cell.

In some embodiments, the HDZip polypeptide is an HDZipI polypeptide as hereinbefore described.

As referred to herein, a “genetically modified cell” comprises a cell that is genetically modified with respect to the wild type of the cell. As such, a genetically modified cell may be a cell which has itself been genetically modified and/or the progeny of such a cell. As such, genetically modified cells include, for example, transgenic cells and mutant cells or the progeny of such cells. Furthermore, the cells of the second aspect of the invention may be isolated single cells, cultured cells or cells in planta.

The plant cells of the second aspect of the invention comprise a modulated rate and/or extent of cell wall deposition relative to an unmodified form of the cell. This should be understood as a difference in the rate and/or extent of cell wall deposition between the genetically modified cell and an unmodified or wild type form of the same cell.

In some embodiments, the rate and/or extent of cell wall deposition in the genetically modified cell may be modulated as described with reference to the method of the first aspect of the invention.

In some embodiments, the cell comprises a modulated rate and/or extent of secondary cell wall deposition.

In some embodiments the cell may comprise increased or decreased expression of an HDZipI polypeptide relative to an unmodified form of the cell, leading to an increased or decreased rate and/or extent of secondary cell wall deposition, respectively.

In some embodiments, the HDZipI polypeptide having modulated expression in the genetically modified cell is as hereinbefore described with reference to the first aspect of the invention.

In some embodiments, the expression of the HDZip polypeptide is modulated by modulating the expression of an HDZip polypeptide-encoding nucleic acid in the plant cell.

Suitable HDZip polypeptide encoding nucleic acids, and methods for modulating their expression in a plant cell, include those hereinbefore described with reference to the first aspect of the invention.

As set out with respect to the first aspect of the invention, the modulated rate and/or extent of cell wall deposition in the cell may include the actual rate and/or extent of cell wall deposition by a plant cell and/or any process in the plant cell which is involved in or associated with cell wall deposition. Thus, any one or more of the following may be modulated in the cell: actual cell wall deposition, the expression of one or more cell wall associated proteins, the amount of one or more primary or secondary cell wall components in the plant cell wall, and the like.

The plant cell of the second aspect of the invention may be any cell from an organism of the kingdom Plantae. As such, the cell may be a bryophyte cell or a vascular plant cell. Generally, the cells used in accordance with the present invention include walled members of this kingdom. However, naturally non-walled members of the kingdom may be used and the present invention may be used to promote primary or secondary cell wall deposition.

In some embodiments, the cell is a monocotyledonous or dicotyledonous angiosperm plant cell or a gymnosperm plant cell. In some embodiments, the monocotyledonous plant cell, a cereal crop plant cell, or a barley plant cell as previously described.

In a third aspect, the present invention provides a plant or a part, organ or tissue thereof comprising one or more cells according to the second aspect of the invention.

A plant of the third aspect of the invention may be any multicellular organism of the kingdom Plantae, including bryophytes and vascular plants. In some embodiments, the plant is a monocotyledonous or dicotyledonous angiosperm plant or a gymnosperm plant. In some embodiments, the plant is a monocotyledonous plant, a cereal crop plant, or a barley plant as previously described.

As referred to herein, “a plant or a part, organ or tissue thereof” should be understood to include a whole plant or any part thereof. As such, this term may encompass whole plants, plant reproductive material or germplasm including seeds, vegetative plant tissue, harvested plant tissue, silage, cuttings, grafts, explants and the like.

As a result of including one or more cells having modulated cell wall deposition and/or expression of an HDZip polypeptide, plants of the third aspect of the invention may exhibit an altered phenotype relative to wild type plants of the same taxon.

Characteristics of the altered phenotype caused by overexpression of HDZipI include, for example, smaller plant size, lighter green colour, early flowering and increased tillering. Further characteristics of the HDZipI overexpression phenotype are described at Table 3 in Example 3.

In light of the above, in a fourth aspect, the present invention also provides a method for altering the phenotype of a plant; the method comprising modulating the expression of a homeodomain/leucine zipper (HDZip) polypeptide in one or more cells of the plant.

In some embodiments, the HDZip polypeptide is an HDZipI polypeptide as hereinbefore described.

In some embodiments, the expression of the class I HDZip polypeptide in the one or more plant cells is increased and this effects an altered phenotype comprising one or more of the characteristics described in Table 3 of Example 3.

The fourth aspect of the invention may be practiced on any suitable plant as hereinbefore described. In some embodiments, the plant is a monocotyledonous plant, a cereal crop plant or a barley plant.

In a fifth aspect, the present invention provides a method for determining and/or predicting the rate and/or extent of cell wall deposition in a plant, or a part, organ, tissue or cell thereof, the method comprising determining the expression of a homeodomain/leucine zipper (HDZip) polypeptide in the plant or a part, organ, tissue or cell thereof.

In some embodiments, the HDZip polypeptide is a class I HDZip polypeptide, as hereinbefore described.

In some embodiments, the method is for determining the rate and/or extent of secondary cell wall deposition in a plant, or a part, organ, tissue or cell thereof.

The method of the fifth aspect of the present invention may be used, among other things, to select a plant, or part, organ, tissue or cell thereof, which has a desired rate and/or extent of cell wall deposition. These plants may then be selected for breeding or other techniques (such as clonal propagation) to generate progeny plants having a desired rate and/or extent of cell wall deposition in one or more cells. In addition, a plant or a part, organ, tissue or cell thereof may be selected for further downstream processing or application on the basis of the determined or predicted rate and/or extent of cell wall deposition.

The method contemplates any means by which the expression of an HDZip polypeptide in a cell may be determined. This includes, for example, methods such as determination of the level and/or activity of an HDZip polypeptide in a cell and/or determining the expression of an HDZip polypeptide encoding nucleic acid in the plant, or a part, organ, tissue or cell thereof.

In some embodiments, the expression of the HDZip polypeptide is determined by determining the expression of an HDZip polypeptide-encoding nucleic acid in the plant or a part, organ, tissue or cell thereof. Suitable HDZip polypeptide encoding nucleic acids include those hereinbefore described.

Methods for determining the level and/or pattern of expression of a nucleic acid or polypeptide are known in the art. Exemplary methods of the detection of RNA expression include methods such as quantitative or semi-quantitative reverse-transcriptase PCR (eg. see Burton et al., Plant Physiology 134: 224-236, 2004), in-situ hybridization (eg. see Linnestad et al., Plant Physiology 118: 1169-1180, 1998); northern blotting (eg. see Mizuno et al., Plant Physiology 132: 1989-1997, 2003); and the like. Exemplary methods for determining the expression of a polypeptide include Western blotting (eg. see Fido et al., Methods Mol Biol. 49: 423-37, 1995); ELISA (eg. see Gendloff et al., Plant Molecular Biology 14: 575-583); immunomicroscopy (eg. see Asghar et al., Protoplasma 177: 87-94, 1994) and the like.

This aspect of the invention may be utilised to select a plant or a part, organ, tissue or cell thereof on the basis of a determined and/or predicted relatively low or relatively high cell wall deposition.

For example, in some embodiments, increased or relatively high expression of an HDZipI polypeptide in the plant, or a part, organ, tissue or cell thereof is indicative of an increased rate and/or extent of cell wall deposition in the plant, or a part, organ, tissue or cell thereof.

Conversely, decreased or relatively low expression of an HDZipI polypeptide in a plant or a part, organ, tissue or cell thereof, may be associated with a relatively low rate and/or extent of cell wall deposition.

In the method of the fifth aspect of the invention, the rate and/or extent of cell wall deposition determined and/or predicted in accordance with the method may include the actual rate and/or extent of cell wall deposition by a plant cell and/or the rate and/or extent of any process in the plant cell which is involved in or associated with cell wall deposition including, for example, the expression of one or more cell wall associated proteins as hereinbefore described, the rate and/or extent of lignin deposition, or a phenotype in a plant associated with cell wall deposition as hereinbefore described.

The method of the fifth aspect of the invention may be practiced on any plant cell, as hereinbefore defined. However, in some embodiments, the cell may be a monocotyledonous or dicotyledonous angiosperm plant cell or a gymnosperm plant cell. In some embodiments the cell is a monocotyledonous plant cell, a cereal crop plant cell or a barley cell.

Although cereal crop plants are suitable monocotyledonous plants, the other monocotyledonous plants may also be used, such as other non-cereal plants of the Poales, specifically including pasture grasses such as Lolium spp.

Finally, reference is made to standard textbooks of molecular biology that contain methods for carrying out basic techniques encompassed by the present invention, including DNA restriction and ligation for the generation of the various genetic constructs described herein. See, for example, Maniatis et al., Molecular Cloning: A Laboratory Manual (Cold Spring Harbor Laboratory Press, New York, 1982) and Sambrook et al. (2000, supra).

The present invention is further described by the following non-limiting examples:

### BRIEF DESCRIPTION OF THE FIGURES

FIG. 1 shows unrooted phylogenetic trees for the TaHDZipI-2 amino acid sequence (SEQ ID NO: 1) with HDZip class I and II proteins from rice (A) and Arabidopsis (B). Proteins grouped together in class I and class II are marked with ovals.

FIG. 2 shows the phenotype of transgenic barley plants with constitutive overexpression of TaHDZipI-2. A—transgene expression levels in different generations of several independent transgenic lines; B—Flowering time of T₁ transgenic plants in days relative to control plants (value 0). X-axis labels show plant lines and sub lines. Wild type plants and transgenic plants transformed with empty vector were used as a control.

FIG. 3 shows the shape, size and colour of spikes and grain in control and transgenic plants. A—mature spikes of control plants (left) and HDZipI plants (right); B—close up of grain from HDZipI plants (upper) and control plants (lower); C—grains from control and different generations of several independent transgenic lines of HDZipI plants in comparison to control grain. Numbers reflect number of lines and sub lines.

FIG. 4 shows the anther morphology and dehiscence in HDZipI plants (T₂) and control plants. Anthers of transgenic plants are smaller in size but can normally open.

FIG. 5 shows the number of trichomes and size of epidermal cells of leaf from control and transgenic plants analyzed using scanning electron microscopy. Trichomes are indicated by arrows in control (A), and HDZipI (B) plants. Cell length in stem epidermis cells of control (C) and HDZipI (D) plants. The beginning and the end of cells are shown with arrows.

FIG. 6 shows differences in stem and leaf morphology and cell number, shape and size in control and transgenic plants. The same stage of plant development (where it was possible), the same leaf number and the same position in the tissue has been used for the comparison of cross sections. Panels C1-C3 show stems of control plants, while C4 and C5 show control plant leaf sections. Stem (B1, B2 and B6), leaf sheath (B3-B5) and leaf (B7-B8) of control (B1), stemless HDZipI transgenic plants with strong (B3-B5, B7, and B9) and moderate (B2, B6, B8, and B10) phenotype. Small diameter of stem (B2), absence of stem (B3 and B4), similarity of vascular tissue in stem and leaf sheath (B5 and B6) and changed morphology of leaf of stemless HDZipI plants (B7 and B9) versus HDZipI plants with moderate phenotype (B8 and B10) can be observed. Magnification is shown in the right lover corner of each picture.

FIG. 7 shows regulation of expression of potential downstream genes in wild type (WT) and T₀-T₂ progeny of transgenic lines. A—Expression of genes, which are known to be involved or can be potentially involved in biosynthesis of secondary cell walls. B—Expression of genes, which are involved or potentially can be involved either in biosynthesis of primary cell walls (HvCesA1, HvCesA3) or lignin biosynthesis (HvLAC1, HvLac2, Hv4CL1, HvOMT1, HvOMT2). C—Expression of some barley orthologs of transcription factors from other plants which have been reported to be involved in regulation of the biosynthesis of secondary cell walls. HvCesA8 and HvCobra5 were used as positive controls. Further details for the tested genes are shown in Table 4.

FIG. 8 shows lignin content and distribution in different tissues of control and transgenic plants. Visualization of lignin in stem, leaf, and leaf sheath (A10) of control (C1—leaf, C2—stem, C3—leaf, C4—stem, C5—stem), and HDZipI (A1—leaf, A2—stem, and A3—leaf sheath) plants using fluorescence under UV light. Redistribution (A6) and higher level of lignin in cells close to the centre of the stem (A8) was detected in HDZipI plants.

FIG. 9 shows the cell wall content and thickness in control and transgenic plants. Serial transverse sections of stem of control (C1-05) and HDZipI (A1-5) plants were either stained with Calcofluore white (C1 and A1) or immunostained with specific monoclonal antibodies against callose (call, C2 and A2) homogalacturonan (partially Me-HG/de-esterified HG) (jim5; C3 and A3), (1→4)-β-D-xylan (Lm10; C4 and A4) and Lm11 (1→4)-β-D-xylan/arabinoxilan (C5 and A5). Transgenic plants have different content of some components and different thickness of cell walls.

FIG. 10 shows transmission electron microscopy of cell walls of stem epidermal cells (B1, B4) and stem vascular bundles (B2, B5) of control (B1, B2) or HDZipI transgenic (B2, B5) plants.

### EXAMPLE 1

### Isolation of Wheat TaHDZipI-2

TaHDZipI-2 cDNA clones were isolated from a cDNA library made from the liquid fraction of wheat endosperm at 3-6 DAP (days after pollination). The clones were identified from a yeast one hybrid screen using a 4× repeat of the nucleotide sequence CAATNATTG as bait (Lopato et al., Plant Methods 2: 3, 2006). Originally each repeat in the bait contained a different nucleotide in the N position. However, it has been demonstrated in Arabidopsis that these classes of transcription factors will bind to the bait sequence irrespective of the base in the central position. Wheat HDZip proteins appear to interact well with the CAATCATTG repeats in the Y1H assay and can be isolated using this repeat.

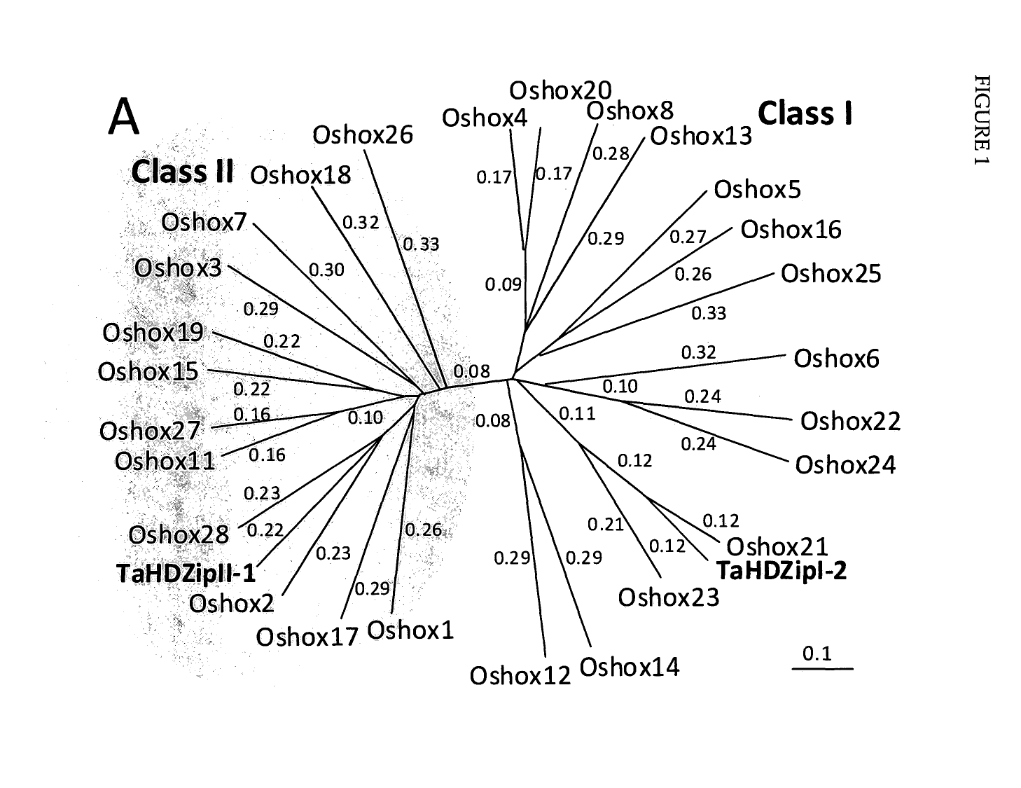

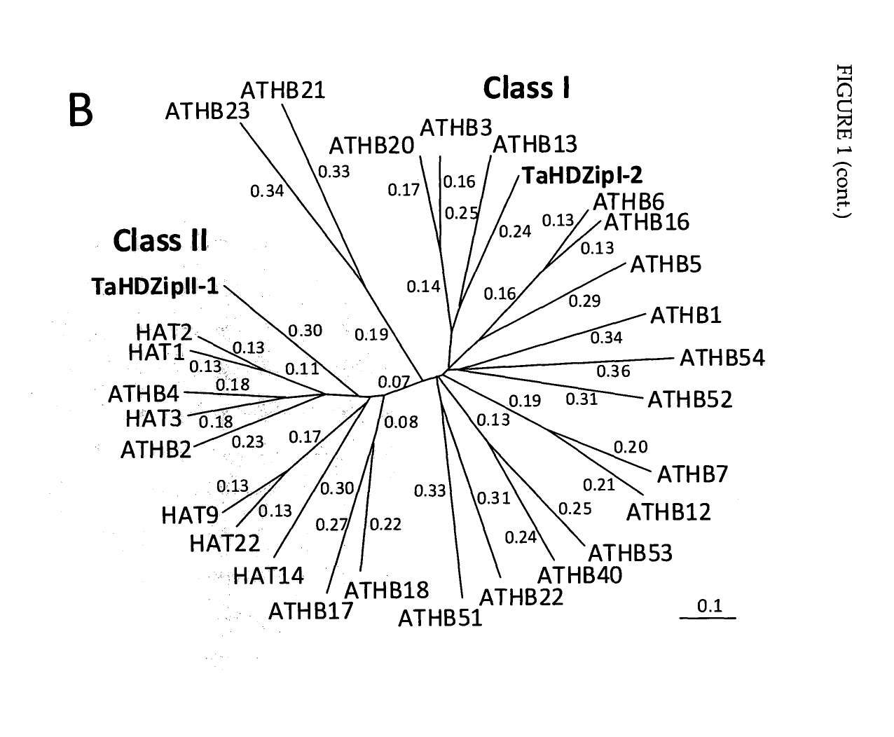

The cloned cDNA was designated and TaHDZipI-2 (SEQ ID NO: 2). Based on a sequence comparison of the encoded amino acid sequence (SEQ ID NO: 1) to HDZip class I and II proteins from rice and Arabidopsis (FIG. 1), TaHDZipI-2 was found to cluster with class I HDZip proteins.

### EXAMPLE 2

### Expression of TaHDZipI-2 in Wheat

Transcripts of TnHDZipI-2 were detected by Q-PCR in flowers, early grain and shoots of seedlings, but were very low in roots of seedlings and young inflorescence. No expression was found in leaves or stems of mature plants.

### EXAMPLE 3

### Phenotype from Over-Expression of TaHDZipI-2 in Barley

Barley (Hordeum vulgare cv. Golden Promise) was transformed with TaHDZipI-2 in a modified of pMDC32 vector, in which 2×35S promoter was replaced with the polyubiquitin promoter from maize. The pUbi vector containing the coding region of TaHDZipI-2 (SEQ ID NO: 2) under the transcriptional control of the polyubiquitin promoter was designated pUbi-TaHDZipI-2.

Plants which were successfully transformed with pUbi-TaHDZipI-2, and which subsequently overexpress HDZipI-2, are referred to herein as “HDZipI plants”.

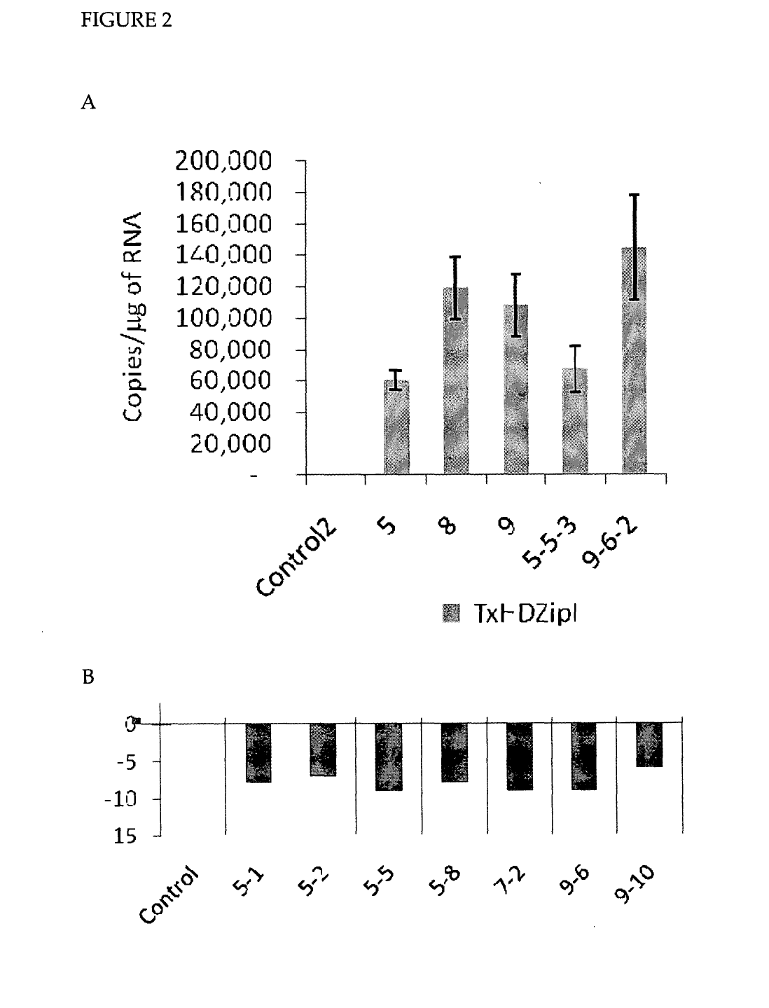

10 independent transgenic lines were produced with HDZipI, all showing expression of the transgene (See FIG. 2). The copy number, level of transgene expression and relative strength of phenotype in T₀ plants is summarized in Table 2, below:

TABLE 2
Transgene incorporation in HDZipI plants

| | | Strength of | |
|---|---|---|---|
| Name of the T₀ | Transgene copy | transgene | Strength of |
| transgenic line | number | expression | phenotype |
| G109-1 | No data | ++ | + |
| G109-2 | No data | ++ | + |
| G109-3 | No data | +++ | ++ |
| G109-4 | No data | ++ | + |
| G109-5 | No data | +++ | ++ |
| G109-6 | No data | ++ | + |
| G109-7 | No data | +++ | +++ |
| G109-8 | No data | +++ | +++ |
| G109-9 | No data | +++ | +++ |
| G109-10 | No data | ++ | ++ |

The ten HDZipI T₀ plant lines showed a characteristic aberrant phenotype: 3 lines showed a strong phenotype (+++), 3 showed an intermediate phenotype (++), and 4 lines had a weak but clear phenotype (+).

The characteristic features of the HDZipI overexpression phenotype included smaller plant size, light green colour, early flowering and shorter life cycle relative to non-transgenic controls. A summary of the characteristics of the phenotype are shown in Table 3 below:

TABLE 3
Phenotype features of HDZipI plants relative to control plants

| | HDZipI-2 overexpression phenotype |
|---|---|
| Feature | relative to control |
| Germination rate | 85% |
| Plant size | Smaller |
| Flowering time | 1-2 weeks earlier |
| Plant colour | Brighter |
| Leaf thickness | Thinner |
| Leaf length | Shorter |
| Stem thickness | Thinner |
| Internode length | Shorter |
| Number of stems | Increased |
| Spike shape | Slightly shorter, thinner |
| Anther size | Smaller |
| Anther dehiscence | Normal |
| Pollen | Normal |
| Sterility | Not observed |
| Grain size | Smaller |
| Grain colour | Brighter |
| Cell length in stem | Slightly decreased |
| Number of trichomes | Strongly decreased |
| Lignin content | Slightly higher, redistributed in leaf |
| Cellulose content | Unclear |
| Cell walls in vascular tissue | Thicker |

T₁ generation HDZipI plants with a strong phenotype showed extreme dwarfism. These plants developed a bushy mass of leaves but neither stem growth nor transition to flowering was observed. The leaves of the plants with a strong phenotype were also pale yellow-green in colour. After one year of no change, the HDZipI plants with a strong phenotype showed signs of senescence and finally died.

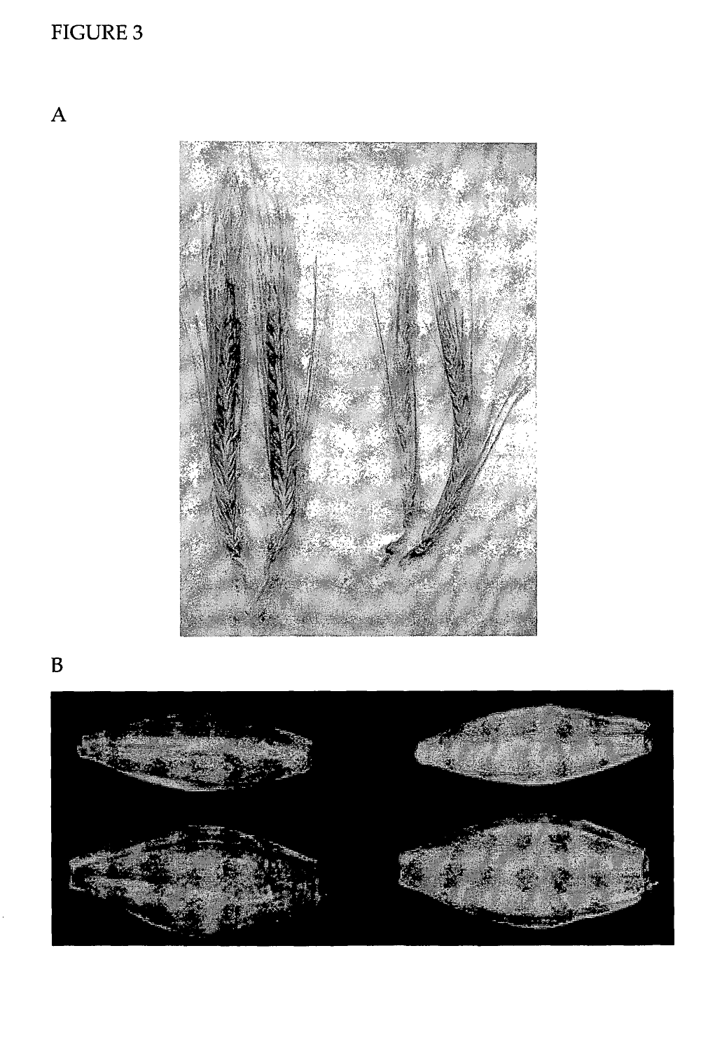

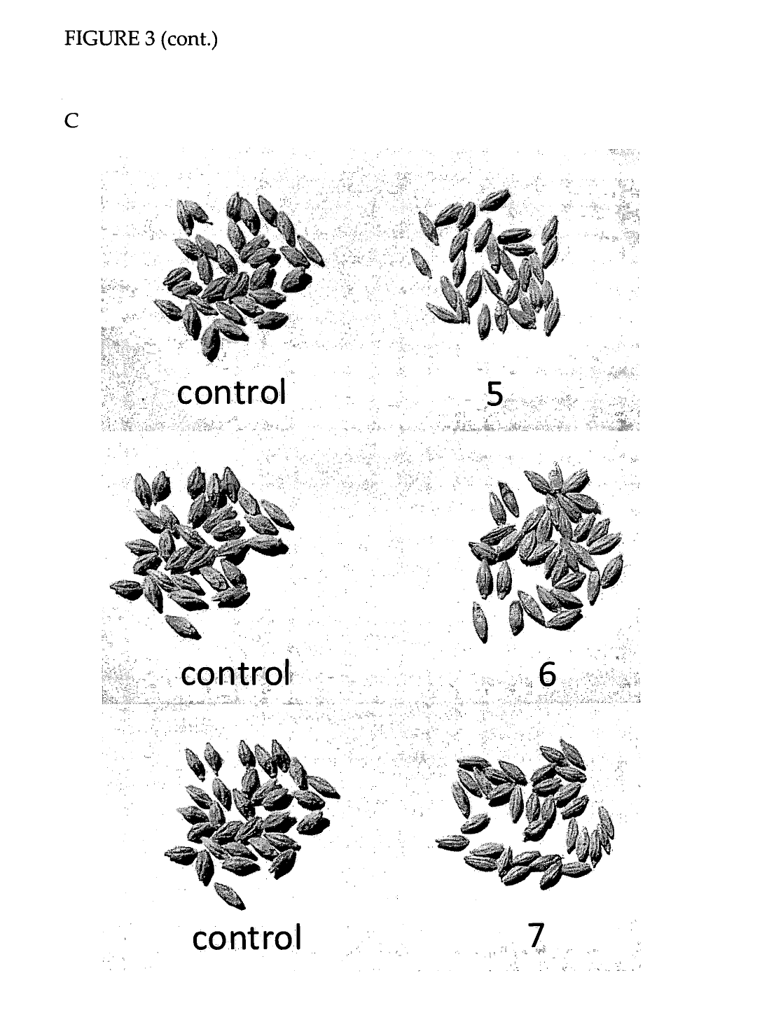

In HDZipI-2 overexpressing transgenic plants with an intermediate or weak phenotype, the number of shoots was 2-3 fold higher than in control plants. The stems of these plants were also thinner than those of controls. The leaves of HDZipI-2 overexpressing transgenic plants with an intermediate or weak phenotype were also smaller and thinner than leaves of controls. The spikes of plants with an intermediate or weak phenotype were smaller, slightly shorter and thinner than spikes of control plants (FIG. 3). The shape and size of spike strongly was observed to correlate with the level of transgene expression.

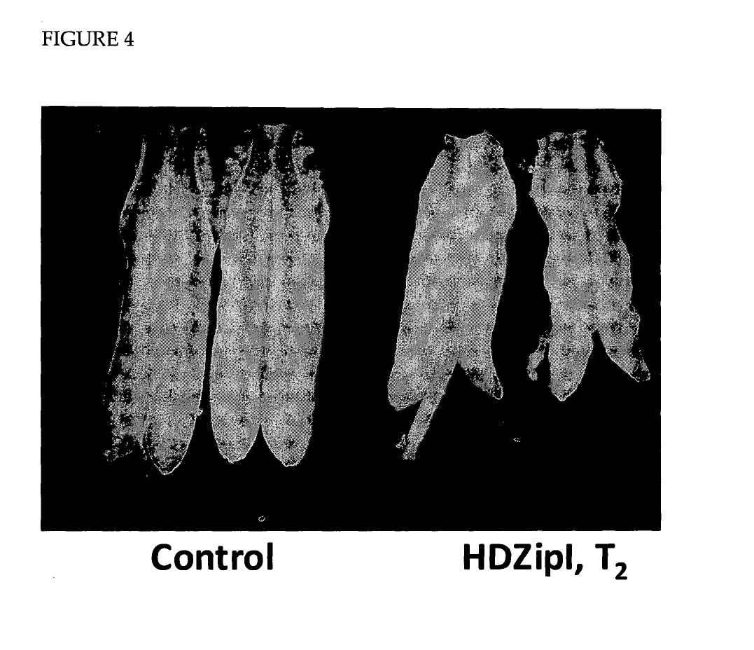

No flower defects were observed in HDZipI plants with an intermediate or weak phenotype, except that the anthers in these transgenic plants were smaller than in control plants (FIG. 4). Grain of HDZipI plants with an intermediate or weak phenotype was smaller (thinner) than control grain and had a light, near white colour (FIG. 3). Grain of HDZipI plants with an intermediate or weak phenotype had a germination rate of about 85%.

### EXAMPLE 4

### Cell Morphology of Transgenic Plants

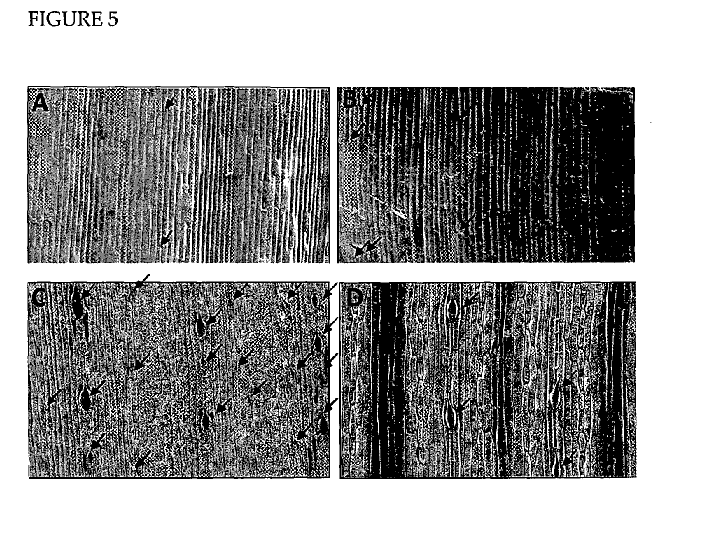

The stem epidermis cells of HDZipI plants were shorter than in control cells (FIG. 5). In addition, the leaves of HDZipI plants contained fewer trichomes than control plants (FIG. 5).

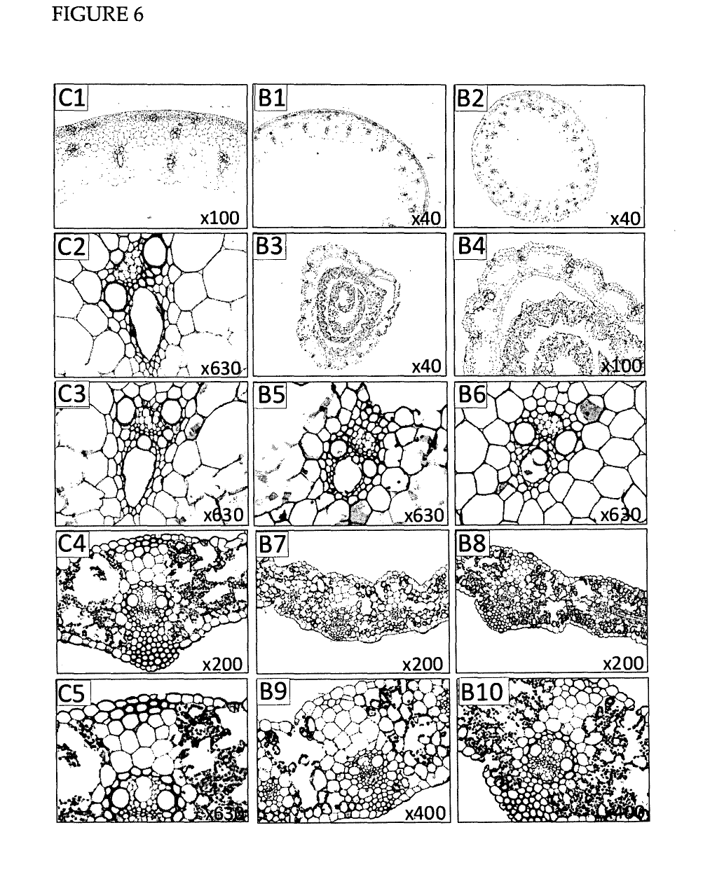

Stem transverse sections of HDZipI plants with an intermediate phenotype show the same number of cell layers as control plants, however, the diameter of stems is much smaller than of control plants (FIG. 6). HDZipI plants with a strong phenotype produced no stem. In these plants cross-sections taken close to the root contained a sheath of several consecutive leaves with strongly changed morphology (FIG. 6).

No morphological changes were observed in vascular tissue of HDZipI transgenic plants with intermediate or weak phenotypes. Also, no visible defects in anther dehiscence or in pollen shape were detected in these HDZipI plants, except the anthers were smaller than control anthers (FIG. 4).

### EXAMPLE 5

### TaHDZipI-2 is a Positive Transcription Regulator of Genes Involved in Secondary Cell Wall Biosynthesis

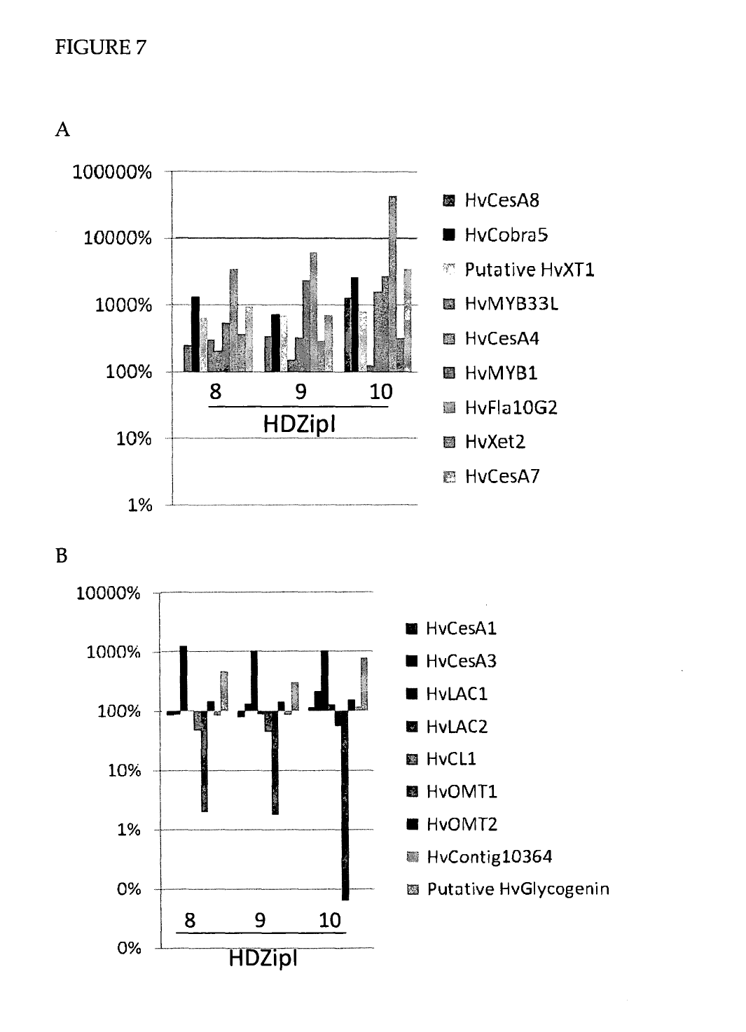

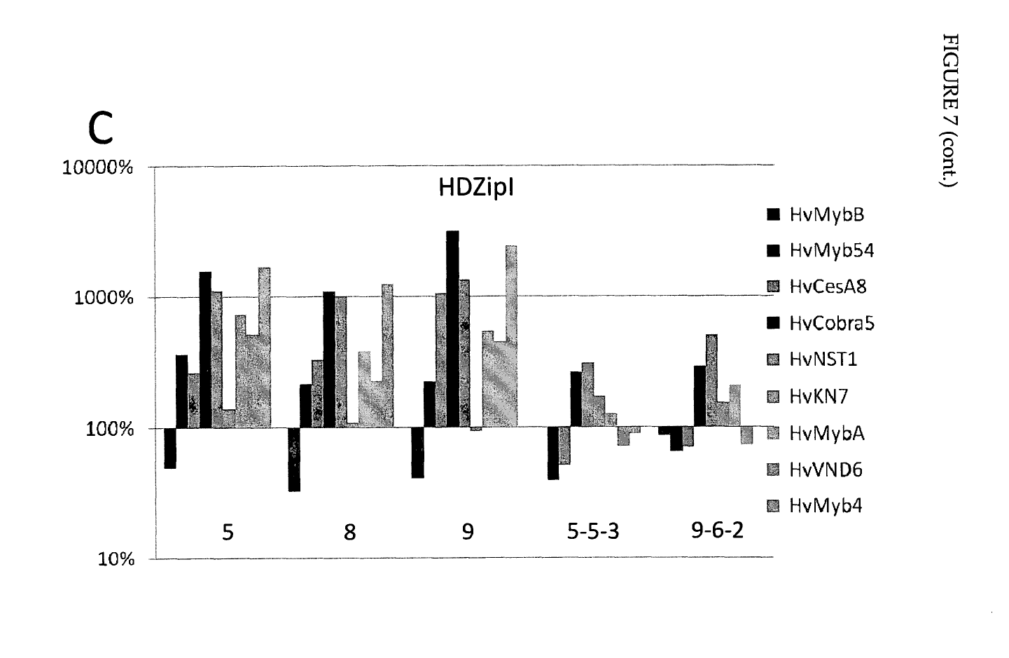

Q-PCR was used to test levels of expression of some genes involved in secondary cell wall biosynthesis in barley. Two groups of co-ordinately transcribed cellulose synthases were identified in barley. One of them contains HvCesA4, HvCesA7 and HvCesA8, which are cellulose synthases are involved in the biosynthesis of secondary cell walls. Expression of these three CesA genes was up-regulated in all tested HDZipI plants (FIG. 7).

Barley oligo arrays were used to identify additional genes which are co-expressed with the three cellulose synthases in different tissues. The identified genes are listed in Table 4, below:

TABLE 4
List of genes co-expressed with HvCesA4/A7/A8.

| | | | Co-expression with |
|---|---|---|---|
| | | Length of | HvCesA7, 8 and 4 in |
| New name | Full name | the gene | different tissues |
| HvCesA8 | Cellulose synthase A8 | Full | + |
| HvCesA7 | Cellulose synthase A7 | Full | + |
| HvCesA4 | Cellulose synthase A4 | Full | + |
| HvCobra5 | COBRA | Full | + |
| HvFLA10G2 | Fasciclin-like | Full | + |
| | arabinogalactan proteins | | |
| HvXET2 | Xyloglucan | Full | + |
| | endotransglycosylase | | |
| HvC19112 | Putative | Partial | + |
| | glycosiltransferase | | |
| HvOMT1 | Caffeic acid 0- | Full | − |
| | methyltransferase | | |
| HvOMT2 | Caffeic acid 0- | Full | − |
| | methyltransferase | | |
| HvLAC2 | Laccase | Partial | + |
| Putative HvXT1 | Putative glycogenase | N/A | + |
| HvLAC1 | Laccase | Full | − |
| HvCesA1 | Cellulose synthase A1 | Full | − |
| HvCesA3 | Cellulose synthase A3 | Full | − |
| HvContig10364 | unknown | Partial | − |
| Hv4CL1 | 4-Coumarate ligase | Full; | − |
| | | contig | |
| Putative HvXT1 | Putative | Partial | |
| | Xylosetransferase | | |
| HvMYB33L | 2,3-MYB transcription | Partial | + |
| | factor | | |
| HvMYB1 | 2,3-MYB transcription | Full | + |
| | factor | | |
| HvMYBB | 2,3-MYB transcription | Full | − |
| | factor | | |
| HvMYB54 | 2,3-MYB transcription | Full | + |
| | factor | | |
| HvMYBA | 2,3-MYB transcription | Full | + |
| | factor | | |
| HvMYB4 | 2,3-MYB Transcription | | |
| | factor | | |
| HvNST1 | NAC transcription factor | Full | + |
| HvVND6 | NAC transcription factor | Full | + |
| HvKN7 | Homeodomain (Knox) | Full | − |
| | transcription factor | | |

Expression of each of these genes was tested in the leaves of control and transgenic plants and the results are shown in FIG. 7. As shown in FIG. 7, most of tested genes, which are known to be related to the secondary cell wall biosynthesis, were up to 100 fold up-regulated in HDZipI plants. Two cellulose synthase genes, HvCesA1 and HvCesA3, which are involved in the primary cell wall biosynthesis, were used as negative controls. Expression of HvCesA1 and HvCesA3 was not substantially affected in the transgenic plants.

FIG. 7 also shows the regulation of additional genes that are potentially related to lignin biosynthesis in HDZipI plants. In some cases, genes were upregulated in the transgenic plants (eg. HvLAC1, HvGlcogenin). However, in some cases, down-regulation of genes in the HDZipI plants was also observed (eg. HvCL1, HvOMT1). The expression of HvOMT2 was not substantially influenced in HDZipI plants.

Several genes encoding transcription factors were identified in barley which are orthologs of previously reported transcription regulators of secondary cell wall and lignin biosynthesis in Arabidopsis and other plants. Expression of six transcription factors was studied using quantitative RT-PCR. As shown in FIG. 7, four transcription factors genes, HvMyb54, HvMybA, HvNST1, and HvVND6, were strongly upregulated in T₀ HDZipI plants. However, in the T₂ generation the level of up-regulation of some downstream genes was two-three fold lower than in T₀ plants, while some genes even became down-regulated.

Expression of HvMYBB was moderately downregulated in T₀ HDZipI plants. However, in T₂ HDZipI plants no effect on the expression of this gene was observed. Transcription of HvKN7 was slightly up-regulated in the transgenic plants. However, up-regulation of this gene was lower in T₀ generation, and increased in the T₂ generation.

### EXAMPLE 6

### Regulation of Lignin and Cellulose Biosynthesis in HDZipI Plants

Since the expression of several enzymes related to secondary cell wall biosynthesis is regulated by TaHDZipI-2, changes in the content of lignin and cellulose, as well as any difference in the thickness of secondary cell walls, in transgenic plants was assessed.

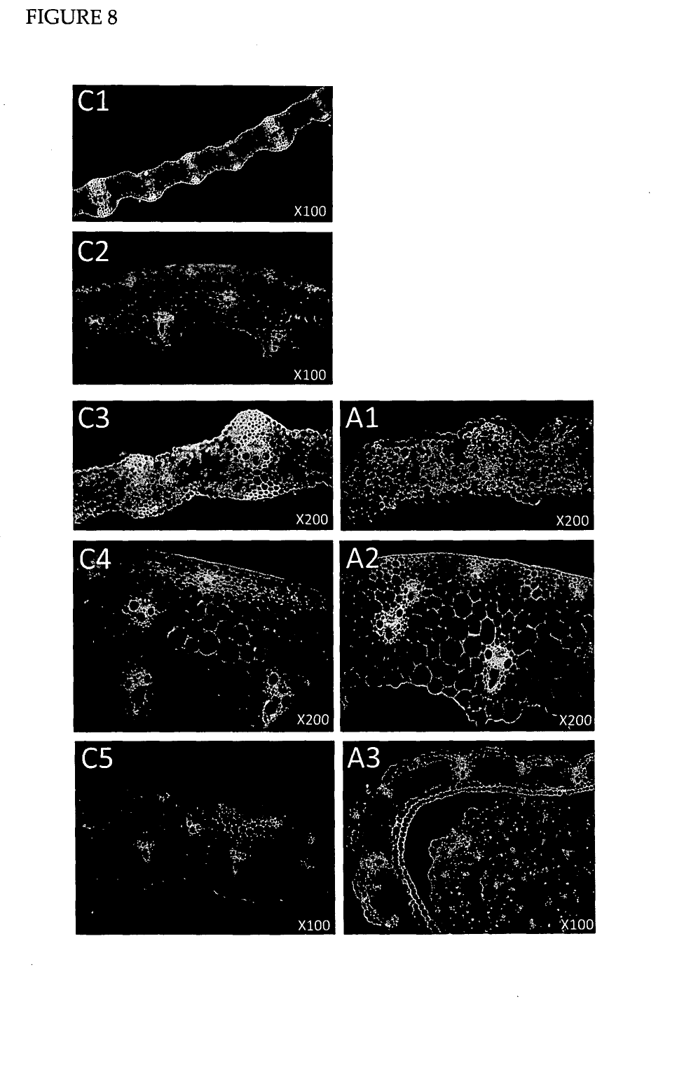

Lignin content was analysed using lignin autofluorescence under UV light (FIG. 8). In HDZipI plants, a re-distribution of lignin was observed in leaf tissues. Lignin content was observed to decrease in epidermis and vascular tissues of leaves, but increase in the rest of the leaf cells. In the stem of plants with a moderate phenotype, a increase in lignin content was observed in vascular tissues and cells, which are situated closer to the middle part of the stem.

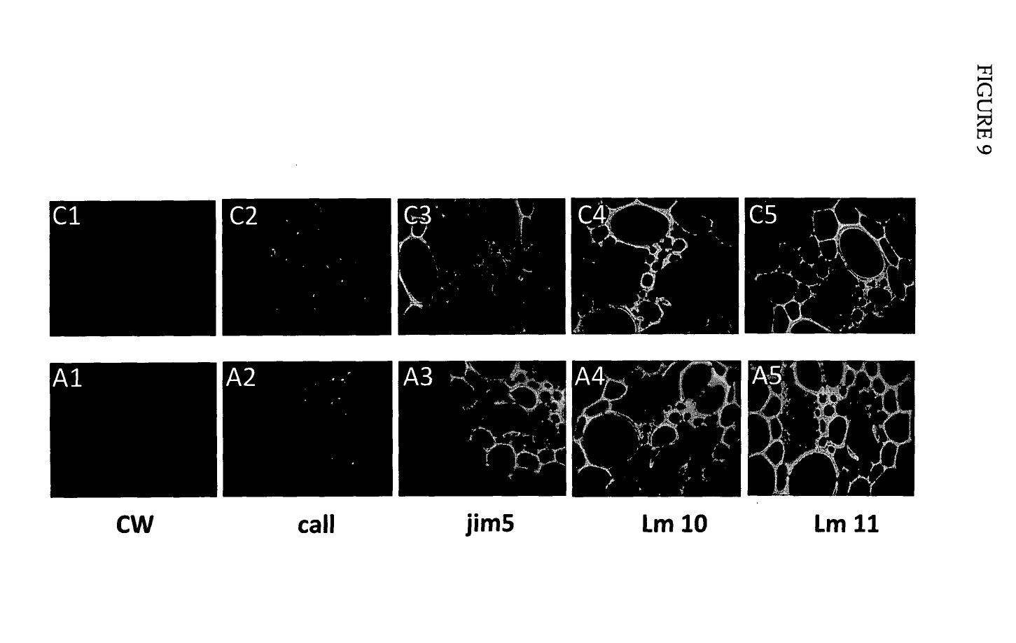

Cell walls in control and transgenic plants were also analysed in cross sections of stem vascular tissue immunostained with specific monoclonal antibodies against callose (call), homogalacturonan (partially Me-HG/de-esterified HG), (1→4)-β-D-xylan, and (1→4)-β-D-xylan/arabinoxilan. The results are shown in FIG. 9. No callose (call) was detected. However, content of pectin (jim5) and xylan/arabinoxylan (Lm10 and Lm11) was elevated in HDZipI overexpressing plants. An increase in the thickness of cell walls for HDZipI overexpressing plants was also observed.

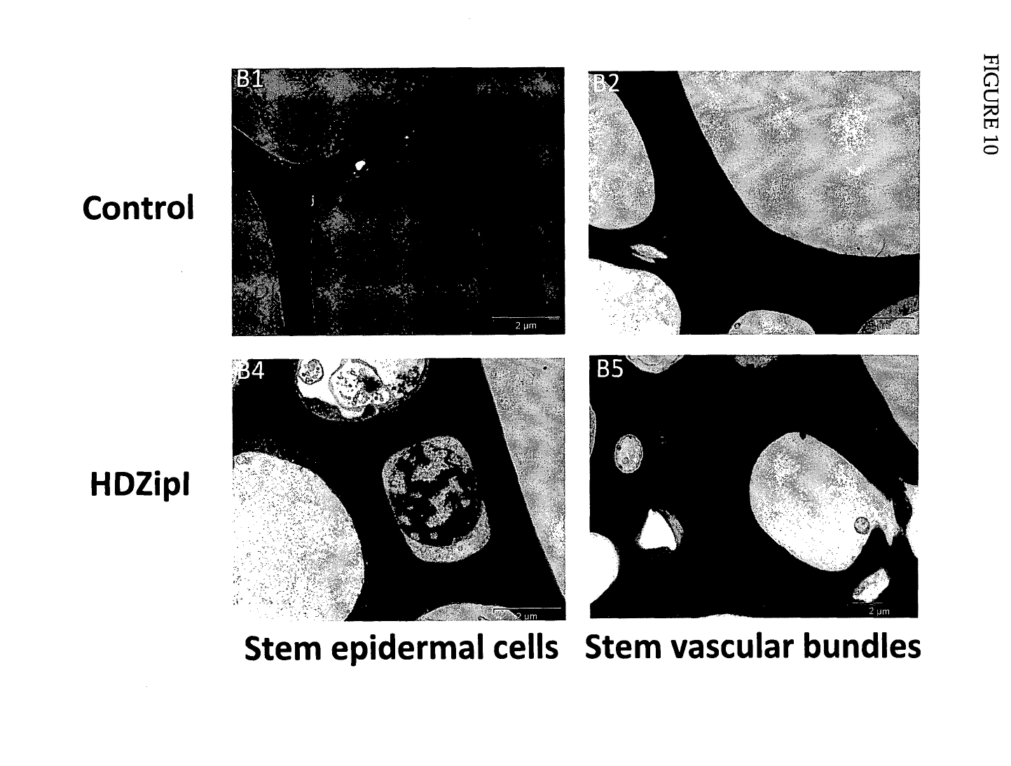

As shown in FIG. 10, a difference in the secondary cell wall of stem epidermal cells and vascular bundles of HDZipI plants and control plants was also detected using TEM.

### EXAMPLE 7

### Discussion

TaHDZipI-2 cDNA was cloned from the liquid fraction of wheat endosperm at 3-6 DAP using a 4× repeat of the nucleotide sequence CAATNATTG as bait.

Sequences of all HDZip class I and II genes from Arabidopsis and rice were identified. TaHDZipI-2 was identified as belonging to class I in the HDZip family (FIG. 1). The closest homologues of TaHDZipI-2 are Oshox21 in rice and ATHB13 in Arabidopsis. Oshox21 and another close homologue of TaHDZipI-2, Oshox23, were found to be mostly expressed in rice seedlings, but were also detected in panicles. These data are in good correlation with the Q-PCR results for the expression of TaHDZipI (Lopato et al., 2006, supra). Thus, at least Oshox21, Oshox23 and ATHB13 may be functionally equivalent to TaHDZipI-2.

Since TaHDZipI-2 was isolated from developing grain, it is expressed in grain and expected to be involved in grain development. Comparison of HDZipI grain colour and size with colour and size of control grain confirms such involvement (see FIG. 3). The white colour and smaller size of HDZipI grain may be the result of early biosynthesis of secondary cell walls in the seed coat which leads to early termination of grain growth, thicker cell walls and additional lignification.

A relatively high level of TaHDZipI-2 expression was detected by Q-PCR in flowers, early grain and shoots of seedlings. However, no expression was observed in green tissues of mature plants (Lopato et al., 2006, supra). Similarity of expression with ATHB13 and changes in leaf shape and size might suggest similar function of these genes.

The expression pattern, multiple changes in leaf morphology and near total inhibition of stem growth in plants with strong overexpression of HDZipI-2 suggest involvement of HDZipI-2 in development of these organs at early stages of seedling growth. Smaller size, early transition to flowering, and early senescence of HDZipI transgenic plants suggest a possible role of HDZipI-2 in the control of the length of some critical phases in plant development. In the case of mild ectopic expression, some phases terminate earlier and this leads to earlier commencement of the following phases of growth and thus a shorter life cycle of the plant. In the case of very strong ectopic expression some phases of plant development terminate prematurely, before minimal development of particular organs (eg. meristems) or cell groups occur, which is important for the transition to the next phase of development. Because such transition(s) become impossible plants with strong HDZipI-2 overexpression phenotypes die after an extended stemless seedling stage without transition to flowering.

Cellulose is a main component of plant cell walls. The orientation of cellulose microfibrils within plant cell wall determines the direction of cell expansion and shape, and therefore determines plant morphology. The levels of expression of genes encoding HvCesA1 and HvCesA3, which are known to be responsible for the cellulose synthesis in primary cell walls, remained unaltered in HDZipI-2 plants (see FIG. 7). These two enzymes provide cellulose for primary cell walls during cell expansion, but they are not involved in biosynthesis of secondary cell walls. The last finding strongly supports the idea that HDZipI-2 regulates time and place of termination of cell expansion, rather than the process of expansion itself.

There are several other groups of genes, which have been recently demonstrated to be involved in secondary cell wall biosynthesis such as CesA4, CesA7 and CesA8. Using barley microarrays several full length barley genes and partial EST contigs were identified, which are co-ordinately expressed with HvCesA4, 7 and 8 in different barley tissues. These genes are listed in the Table 4. One of them is HvCobra5, which encodes a glycosylphosphatidylinositol-anchored COBRA-like protein. The HvCobra1 is a close homologue of BRITTLE STALK2 (Bk2) from maize and BRITTLE CULM1 (Bc2) from rice. Bk2 is co-expressed with ZmCesA10, ZmCesA11 and ZmCesA12 genes, which are known to be involved in secondary wall formation; secondary cell wall composition was significantly altered in Bk2 mutant. Both Bk2 and Bc2 were shown to be important for the mechanical properties of maize and rice tissues. It has been demonstrated that HvCobra5 is strongly up-regulated in HDZipI-2 overexpressing plants (see FIG. 7).

Another gene, HvFLA10, which encodes another glycosylphosphatidylinositol-anchored protein from the subfamily of fasciclin-like arabinogalactan proteins (AGP), is also co-expressed with above described group of genes in tissue series of barley oligoarray data. Fasciclin-like arabinogalactan proteins (FLAs) are a subclass of AGPs that have, in the addition to predicted AGP-like glycosylated regions, putative cell adhesion domains known as fasciclin domain. It was found that a homolog of HvFLA10 from Zinnia is expressed in differentiating xylem vessels with reticulate type wall thickening and adjacent parenchyma cells of stem bundles that suggest involvement of this gene in the secondary wall deposition in metaxylem. HvFLA10 demonstrated the same pattern of expression in HDZipI plants as genes of secondary cell wall cellulose synthases and Cobra5 (see FIG. 7).

Another gene co-ordinately expressed with HvCesA4, 7 and 8 from barley is HvXET2, which encodes a xyloglucan endotransglycosylase (XET). Expression of some XET genes is present in tissues in which cell expansion has ceased and it has been demonstrated that XETs have an important role in restructuring of primary walls at the time when secondary cell wall layers are deposited. HvXET2 is up-regulated in HDZipI plants. Similar regulation in HDZipI plants was also detected for a gene encoding a putative xylosyl transferase (Contig19112) for which only partial sequence has been recovered (see FIG. 7).

Genes for two MYB factors, HvMYB1 and HvMYB33L, were identified which have similar spatial pattern of expression to HvCesA8. Both genes were up-regulated in HDZipI plants (see FIG. 7). More barley transcriptional factors were identified in databases using protein sequence homology to reported transcriptional regulators, MybA, MybB, Myb54, NST1, VND6, and KN7. The barley homologues were designated as HvMYBA, HvMYBB, HvMYB54, HvNST1, HvVND6, and HvKN7. Expression of each of these genes, other than HvMYBB and HvKN7, was upregulated in HDZipI plants.

Regulation of several MYB and NAC transcription factors by HDZip factors suggests the high position of HDZipI in the signal transduction pathway, for delivering environmental stimuli signals to genes involved in plant growth and development. HDZipI-2 was demonstrated to regulate the expression of several transcription factors, homologues of which were shown to be involved in transcription regulation of cell wall biosynthesis. This suggests that in at least some cases, HDZipI-2 may control a plant phenotype by indirect regulation of downstream genes via one or more downstream transcription factors.

It is interesting that not all tested genes were upregulated by overexpression of HDZipI-2 (see FIG. 7). However, it is possible that the unexpected regulation of some downstream genes in plants overexpressing HDZipI-2 may be the result of a compensational reaction in the plant to strong and potentially destructive phenotypic changes caused by strong overexpression of HDZipI-2, rather than direct or indirect regulation by TaHDZipI-2.

Normalization of expression of some genes in the third generation of HDZipI plants (see FIG. 7) demonstrates that alternative mechanisms of regulation of genes may exist, and these mechanisms may modulate the phenotype caused by overexpression of HDZipI-2 and provide partial normalization of the plant phenotype even under high levels of transgene expression.

It has been demonstrated that HDZipI-2 can bind the same cis-element as another transcription factor from wheat, HDZipII-1. Therefore, TaHDZipII-1 and TaHDZipI-2 may be negative and positive regulators of the group of genes involved in the same process, although they most probably regulate these genes in different tissues or groups of cells and regulation is initiated by different internal or external stimuli. Since most if not all HDZip transcription factors from class I and II bind the same cis-element, it can be postulated that all members of these two families function in a similar way as regulators of cell expansions in different tissues or cell groups in response to different external and internal stimuli related to requirements of cell expansion and therefore of plant growth, like quality of light, water deficiency, sugar content and concentration of auxin.

In conclusion, it is proposed that HDZipI-2 may be a ‘muster regulator’, which either directly, or indirectly through downstream transcription factors, regulates secondary cell wall initiation and formation in the response on environmental stimuli.

### EXAMPLE 8

### Materials and Methods

### Plasmid Construction

The full-length coding region (CDS) of the TaHDZipI-2 cDNA (Acc No DQ353856) (Lopato et al., Plant Methods 2: 3-17, 2006) was cloned into the donor vector pENTR-D-TOPO (Invitrogen). The cloned insert was sequenced and re-cloned by recombination into the pMDCUbi (pUbi) vector. pUbi is a derivative of pMDC32 vector (Curtis and Grossniklaus, Plant Physiol. 133: 462-469, 2003) in which 2×35S promoter was cut out using HindIII and KpnI restriction sites and replaced with maize polyubiquitin promoter (Christensen et al., Plant Mol Biol. 18(4):675-89, 1992). The resulting construct was designated pUbi-TaHDZipI-2 and was transformed into the Agrobacterium tumefaciens strain AGL1 by electroporation.

The presence of the plasmid in selected bacterial clones was confirmed by PCR using specific primers (Table 2—see later) derived from the CDS of the plant gene.

### EXAMPLE 9

### Materials and Methods

### Plant Transformation and Growth Conditions

Bread wheat (Triticum aestivum L. cv. Chinese Spring) and barley (Hordeum vulgare L. cv. Golden Promise) plants were grown in glasshouse with day temperatures of 18-25° C. and night temperatures of 18-21° C., with a 10-13 h photoperiod.

Construct, pUbi-TaHDZipI-2 was transformed into barley (Hordeum vulgare L. cv. Golden Promise) using an Agrobacterium tumefaciens-mediated transformation as developed by (Tingay et al., Plant Journal 11: 1369-1376, 1997) and modified by (Matthews et al., Molecular Breeding 7: 195-202, 2001). Transgenic plants were grown in a PC2 glasshouse with 10-hr light photoperiod. Plant phenotype was studied in T₀, T₁ and T₂ generations of several independent transgenic lines.

### EXAMPLE 10

### Materials and Methods

### mRNA Isolation and Hybridization Techniques

Transgene integration in some barley lines was confirmed by Southern blot hybridization. Genomic DNA from selected barley lines was digested with Xho1 and probed with the coding sequence of hygromycin phosphotransferase.

Total RNA was isolated from wheat and barley samples using TRI REAGENT (Molecular Research Centre, Inc., Cincinnati, Ohio) and used in Northern blot hybridization as described elsewhere (Sambrook et al., Molecular Cloning: a Laboratory Manual 2^{nd} Ed. Cold Spring Harbor Laboratory Press: Cold Spring Harbor, N.Y., USA, 1989). Pools of single strand cDNAs for Q-PCR were prepared using SuperScript III reverse transcriptase (Invitrogen).

### EXAMPLE 11

### Materials and Methods

### Quantitative PCR Analysis

The forward primer for TaHDZipI-2 was designed for the wheat variety Chinese Spring using the 3′ end of the coding region of the cDNA. T₀ provide specific recognition of transgene cDNA the reverse primer sequence was selected from Nos terminator of vector plasmid. The primer pairs for cell wall enzymes were designed for the barley variety Golden Promise using 3′UTR sequences (Table 2).

TABLE 2
Q-PCR primers

| Target | Forward primer | Reverse primer |
|---|---|---|
| TaHDZipI-2 | CAGCTTCGGCAACCTGCTGTG | TTGCCAAATGTTTGAACGATC |
| | (SEQ ID NO: 3) | (SEQ ID NO: 4) |
| Laccase 1 | TCATTGCCAGAGTGTTGTCAG | CTAGGCTTTATTTAGCGATAC |
| | (SEQ ID NO: 5) | (SEQ ID NO: 6) |
| Laccase 2 | TTCCTCCCCCTCCCGAAGATC | AAGAACGTATTTCCGCTATTC |
| | (SEQ ID NO: 7) | (SEQ ID NO: 8) |
| HvCesA4 | GCCCAAGGGACCCATTCTTA | TTAGAACTTGGAACCCCCCA |
| | (SEQ ID NO: 9) | (SEQ ID NO: 10) |
| HvCesA7 | TGAGCAGCTGCCGTTGCTTGG | AATAGTAGCCTACATCACCTCTG |
| | (SEQ ID NO: 11) | (SEQ ID NO: 12) |
| HvCesA8 | ACAGTTTGGACGCAAGTTTTGTATT | CGGTCCTCTGTTCAATTCTTGTTTA |
| | (SEQ ID NO: 13) | (SEQ ID NO: 14) |
| HvCesA1 | TGTGGCATCAACTGCTAGGAAA | CGTACAAAGTGCCTCATAGGAAA |
| | (SEQ ID NO: 15) | (SEQ ID NO: 16) |
| HvCesA3 | ACACGAGTCACTGGGCCAGA | CTGGTAAACTAGTCACCCGCTGA |
| | (SEQ ID NO: 17) | (SEQ ID NO: 18) |

The Q-PCR amplification was performed in a RG 2000 Rotor-Gene Real Time Thermal Cycler (Corbett Research, NSW, Australia) using QuantiTect SYBR Green PCR reagent (Qiagen, VIC, Australia), as described by Burton et al. (Burton et al., Plant Physiol. 134(1): 224-36, 2004). The Rotor-Gene V4.6 software (Corbett Research) was used to determine the optimal cycle threshold (CT) from dilution series and the mean expression level and standard deviations for each set of four replicates for each cDNA were calculated.

### EXAMPLE 12

### Materials and Methods

### Microscopy

Stems and leaves of control and transgenic plants were fixed in 0.25% glutaraldehyde, 4% paraformaldehyde, 4% sucrose and 1 M sodium phosphate. After fixation, plant material was rinsed (2-3 changes in 8 hours to remove fixative) in 1 M sodium phosphate. Tissues were rinsed and dehydrated in a successive ethanol series (70, 90, 95, 100%), and infiltrated step-wise with xylene (25, 50, 75, 100% in ethanol). 7 μm thick sections were stained with 0.01% (w/v) toluidine blue in 0.1% aqueous sodium tetraborate for 1-5 mins.

For lignin autofluorescence observation, semi-thin sections (7 μM thick) were prepared on glass slides and imaged under Laser Dissection microscope (Leica AS LMD). A filter BP 355-425 nm was used as excitation filter and fluorescence was detected at >470 nm.

### EXAMPLE 13

### Materials and Methods

### Transmission Electron Microscopy (TEM)

Samples of leaf and stem from control and transgenic plants were fixed in 4% paraformaldehyde/0.25% glutaraldehyde in PBS, +4% sucrose, pH7.2 and infiltrated with epoxy resin (Procure/Araldite Embedding Kit from ProSeiTech). After embedding blocks were polymerized overnight at 70° C. Using a Rerchert ultramicrotome 1 μm thick survey sections were cut and stained with toluidine blue. Ultrathin (70 nm) sections were cut and mounted on copper/palladium grids. Sections were stained with uranyl acetate and lead citrate and examined in a Philips CM100 Transmission Electron Microscope.

### EXAMPLE 14

### Materials and Methods

### Immunomicroscopy

200 nm thick resin sections were mounted on poly-L-lysine slides and dried overnight at 42° C. Sections were drawn a moat around using a PAP pen and washed in 1×PBS twice for 10 min each. Then incubated with 0.05M glycine in 1×PBS for 20 mins to inactivate residual aldehyde groups and washed with Incubation Buffer (1% BSA in 1× PBS) 2×10 min. Primary antibody against xylan (LM10), arabinoxylan (LM11), pectin (JIM5) and callose kindly provided by Paul Knox Cell Wall Lab (http://www.bmb.leeds.ac.uk/staff/jpk/antibodies.htm) were used followed by incubation with 1:600 dilution of secondary antibody (anti-rat conjugated to Alexa 488) for 1 hours at RT. Sections were counterstained with 0.1% calcofluor white rinsed in water, mounted in Citifluor and coverslip. Sections were examined using Olympus Vanox AHT3 microscope, filter sets for 488 and UV.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

Also, it must be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

1-36. (canceled)

37. A method for modulating the rate and/or extent of cell wall deposition in a plant cell, the method comprising modulating the expression of a homeodomain/leucine zipper (HDZip) polypeptide in the plant cell.

38. The method of claim 37 wherein the HDZip polypeptide is an HDZipI polypeptide.

39. The method of claim 38 wherein the HDZipI polypeptide is a polypeptide comprising the amino acid sequence set forth in SEQ ID NO: 1 or a functional equivalent thereof.

40. The method of claim 37 wherein the cell wall deposition comprises secondary cell wall deposition.

41. The method of claim 37 wherein an increase in the expression of an HDZipI polypeptide in the plant cell effects an increase in the rate and/or extent of cell wall deposition in a plant cell.

42. The method of claim 37 wherein the plant cell is a monocotyledonous plant cell.

43. A genetically modified plant cell comprising a modulated rate and/or extent of cell wall deposition relative to an unmodified form of the cell, wherein modulation of the rate and/or extent of cell wall deposition is effected by modulation of the expression of an HDZip polypeptide in the genetically modified cell, relative to an unmodified form of the cell.

44. The genetically modified plant cell of claim 43 wherein the HDZip polypeptide is an HDZipI polypeptide.

45. The genetically modified plant cell of claim 44 wherein the HDZipI polypeptide is a polypeptide comprising the amino acid sequence set forth in SEQ ID NO: 1 or a functional equivalent thereof.

46. The genetically modified plant cell of claim 43 wherein the cell wall deposition comprises secondary cell wall deposition.

47. The genetically modified plant cell of claim 43 wherein the expression of an HDZipI polypeptide is increased in the genetically modified plant cell and this affects an increase in the rate and/or extent of cell wall deposition in a plant cell.

48. The genetically modified plant cell of claim 43 wherein the plant cell is a monocotyledonous plant cell.

49. A plant or a part, organ or tissue thereof comprising one or more genetically modified plant cells of claim 43.

50. The plant or a part, organ or tissue thereof of claim 49 wherein the plant or a part, organ or tissue thereof is a monocotyledonous plant or a part, organ or tissue thereof.

51. The plant or a part, organ or tissue of claim 49 wherein expression of a HDZipI polypeptide is increased in one of more cells of the plant or a part, organ or tissue thereof and wherein the plant or a part, organ or tissue thereof exhibits an altered phenotype relative to an unmodified form of the plant.

52. A method for altering the phenotype of a plant, the method comprising modulating the expression of a homeodomain/leucine zipper (HDZip) polypeptide in one or more cells of the plant.

53. The method of claim 52 wherein the polypeptide comprises an HDZipI polypeptide.

54. The method of claim 53 wherein the HDZipI polypeptide is a polypeptide comprising the amino acid sequence set forth in SEQ ID NO: 1 or a functional equivalent thereof.

55. The method of claim 52 wherein the expression an HDZipI polypeptide is increased.

56. The method of claim 52 wherein the plant is a monocotyledonous plant.

57. A method for determining and/or predicting the rate and/or extent of cell wall deposition in a plant, or a part, organ, tissue or cell thereof, the method comprising determining the expression of a homeodomain/leucine zipper (HDZip) polypeptide in the plant or a part, organ, tissue or cell thereof.

58. The method of claim 57 wherein the HDZip polypeptide is an HDZipI polypeptide.

59. The method of claim 58 wherein the HDZipI polypeptide is a polypeptide comprising the amino acid sequence set forth in SEQ ID NO: 1 or a functional equivalent thereof.

60. The method of claim 57 wherein the cell wall deposition comprises secondary cell wall deposition.

61. The method of claim 57 wherein increased expression of an HDZipI polypeptide in the plant, or a part, organ, tissue or cell thereof is indicative of an increased rate and/or extent of cell wall deposition in the plant, or a part, organ, tissue or cell thereof.

62. The method of claim 57 wherein the plant cell is a monocotyledonous plant cell.
